(19) United States
(12) Patent Application Publication
(10) Pub. No.: US 2022/0119827 A1
(43) Pub. Date: Apr. 21, 2022

(54) GENOME EDITING TO INCREASE SEED PROTEIN CONTENT

(71) Applicant: PIONEER HI-BRED INTERNATIONAL, INC., JOHNSTON, IA (US)

(72) Inventors: ZHAN-BIN LIU, CLIVE, IA (US); BO SHEN, JOHNSTON, IA (US)

(73) Assignee: PIONEER HI-BRED INTERNATIONAL, INC., JOHNSTON, IA (US)

(21) Appl. No.: 17/286,173

(22) PCT Filed: Oct. 30, 2019
(86) PCT No.: PCT/US2019/058747

§ 371 Date: Apr. 16, 2021

Related U.S. Application Data
(60) Provisional application No. 62/753,628, filed on Oct. 31, 2018.

(51) Int. Cl. C12N 15/82; C12N 9/22; C12N 15/11

(52) U.S. Cl. CPC C12N 15/8213; C12N 15/8251; C12N 2800/80; C12N 15/11; C12N 2310/20; C12N 9/22

(57) ABSTRACT

Soybean seeds with increased protein or oil and having a modified CCT-domain protein or modified expression of a CCT-domain protein are provided. Methods for modifying expression of CCT-domain polypeptides and polynucleotides include genome editing to modify the transcription regulatory region or sequence encoding the CCT-domain polypeptide and transformation with recombinant DNA constructs to enhance or suppress expression.

### CROSS-REFERENCE TO RELATED APPLICATIONS

This application claims the benefit of priority to U.S. Patent Application No. 62/753,628, filed Oct. 31, 2018, the entire contents of which are incorporated by reference.

### REFERENCE TO SEQUENCE LISTING SUBMITTED ELECTRONICALLY

The official copy of the sequence listing is submitted electronically via EFS-Web as an ASCII formatted sequence listing with a file named “7835USPSP_SeqList_ST25” created on Oct. 26, 2018, and having a size of 70 kilobytes and is filed concurrently with the specification. The sequence listing contained in this ASCII formatted document is part of the specification and is herein incorporated by reference in its entirety.

### BACKGROUND

Soybeans are a major agriculture commodity in many parts of the world, and are a source of useful products, such as protein and oil, for human and animal consumption. A valuable product obtained from processed soybeans is soybean meal, which contains a high proportion of protein and is primarily used as a component in animal feed. Soy meal can be further processed to produce soy protein isolates, soy flour or soy concentrates, which can be used in foods, glues and as emulsifiers and texturizers. Soybean plants which produce seeds higher in protein content may contribute to a higher-value crop.

### SUMMARY

Provided are methods for increasing protein content in the seed of a soybean plant by introducing a modification into a CCT-domain gene in a soybean plant and growing the plant to produce a seed, wherein the protein content is increased in the seed, compared to a control seed of a control plant not comprising the modification. The modification can include one or more of (a) a deletion of nucleotides on chromosome 20 in a genomic sequence encoding a polypeptide comprising an amino acid sequence that is at least 95% identical to SEQ ID NO: 2, which results in a modified genomic sequence on chromosome 20 that encodes a polypeptide comprising an amino acid sequence that is at least 95% identical to SEQ ID NO: 4 or 25, such as (i) a deletion of at least 312 and less than 330 nucleotides from position 6003 to 6358 of SEQ ID NO: 9 or (ii) a deletion corresponding to position 6029 to 6349 of SEQ ID NO: 9 or position 6012 to 6332 of SEQ ID NO: 9; (b) a modification of a transcription regulatory sequence of a nucleotide sequence on chromosome 10 encoding a polypeptide comprising an amino acid sequence that is at least 95% identical to SEQ ID NO: 6, such as an insertion of a promotor-enhancer element, an alteration of a repressor element, or a rearrangement of regulatory elements, which results in an increase in expression of the polypeptide; (c) the deletion of part (a) and a second modification of a transcription regulatory sequence of the genomic sequence encoding a polypeptide comprising an amino acid sequence that is at least 95% identical SEQ ID NO: 4 or 25, such as an insertion of a promotor-enhancer element, an alteration of a repressor element, or a rearrangement of regulatory elements, which results in an increase in expression of the polypeptide comprising an amino acid sequence that is at least 95% identical SEQ ID NO: 4 or 25; (d) a modification of one or more nucleotides on chromosome 20 in (i) a polynucleotide encoding a polypeptide comprising an amino acid sequence that is at least 95% identical to SEQ ID NO: 2 or (ii) a transcription regulatory sequence of the polynucleotide, such as (i) an alteration of the polynucleotide resulting in a frame-shift of the polypeptide coding sequence, or (ii) a disruption of a promoter-enhancing element, an insertion of a repressor element or a rearrangement of regulatory elements, which results in suppression of expression of the polypeptide; and (e) a modification of one or more nucleotides on chromosome 10 in (i) a polynucleotide encoding a polypeptide comprising an amino acid sequence that is at least 95% identical to SEQ ID NO: 6 or (ii) a transcription regulatory sequence of the polynucleotide, such a modification resulting in (A) an alteration of the polynucleotide resulting in a frame-shift of the polypeptide coding sequence, or (B) a disruption of a promoter-enhancing element, an insertion of a repressor element, or a rearrangement of regulatory elements, such that the modification results in suppression of expression of the polypeptide. The methods may include, for example, the modifications of parts (a) and (b) or the modifications of parts (b) and (c).

Methods are provided for crossing a plant grown from seed comprising the modified CCT-domain polypeptide with a second different plant and harvesting the progeny seed. In some embodiments the deletion or modification is introduced through targeted DNA breaks.

Plants and seeds having increased protein content are provided, the plants or seeds contain a modified CCT-domain genomic sequence, the modification selected from (a) a deletion of nucleotides on chromosome 20 in a genomic sequence encoding a polypeptide comprising an amino acid sequence that is at least 95% identical to SEQ ID NO: 2, which results in a modified genomic sequence on chromosome 20 that encodes a polypeptide comprising an amino acid sequence that is at least 95% identical to SEQ ID NO: 4 or 25, such as (i) a deletion of at least 312 and less than 330 nucleotides from position 6003 to 6358 of SEQ ID NO: 9 or (ii) a deletion corresponding to position 6029 to 6349 of SEQ ID NO: 9 or position 6012 to 6332 of SEQ ID NO: 9, wherein the plant produces seeds having an increased protein content relative to a control seed not comprising the deletion and a yield that is, for example, at least 80%, 90%, 95%, 100%, 110% or 120% of soybean variety 93B83 when grown under the same environmental conditions; (b) a modification of a transcription regulatory sequence of a nucleotide sequence on chromosome 10 encoding a polypeptide comprising an amino acid sequence that is at least 95% identical to SEQ ID NO: 6, such as an insertion of a promotor-enhancer element, an alteration of a repressor element, or a rearrangement of regulatory elements, which results in an increase in expression of the polypeptide, which results in an increase in expression of the polypeptide, wherein the plant produces seeds having increased protein content relative to a control seed not comprising the modification; (c) the modification of step (a) and a second modification of a transcription regulatory sequence of the genomic sequence encoding a polypeptide comprising an amino acid sequence that is at least 95% identical SEQ ID NO: 4 or 25, such as an insertion of a promotor-enhancer element, an alteration of a repressor element, or a rearrangement of regulatory elements, the second modification resulting in an increase in expression of the polypeptide comprising an amino acid sequence that is at least 95% identical SEQ ID NO: 4 or 25, wherein the plant produces seeds having increased protein content relative to a control seed not comprising the modifications; (d) a modification of one or more nucleotides on chromosome 20 in (i) a polynucleotide encoding a polypeptide comprising an amino acid sequence that is at least 95% identical to SEQ ID NO: 2 or (ii) a transcription regulatory sequence of the polynucleotide, such as (i) an alteration of the polynucleotide resulting in a frame-shift of the polypeptide coding sequence, or (ii) a disruption of a promoter-enhancing element, an insertion of a repressor element or a rearrangement of regulatory elements, such that the modification results in suppression of expression of the polypeptide, wherein the plant produces seeds having increased protein relative to a control seed not comprising the modification; or (e) a modification of one or more nucleotides on chromosome 10 in (i) a polynucleotide encoding a polypeptide comprising an amino acid sequence that is at least 95% identical to SEQ ID NO: 6 or (ii) a transcription regulatory sequence of the polynucleotide, such a modification resulting in (A) an alteration of the polynucleotide resulting in a frame-shift of the polypeptide coding sequence, or (B) a disruption of a promoter-enhancing element, an insertion of a repressor element, or a rearrangement of regulatory elements, such that the modification results in suppression of expression of the polypeptide, wherein the plant produces seeds having increased oil relative to a control seed not comprising the modification.

In some embodiments, methods of plant breeding are provided in which the modified plants or seeds are crossed with a second soybean plant, such as with other modified plants or seeds, to produce progeny seed. Progeny seed produced by the methods which comprise the modification and have increased protein content relative to a control progeny seed not comprising the modification are provided.

In some embodiments, recombinant DNA constructs are provided which comprising a heterologous promoter sequence, such as a weakly expressed or seed-specific promoter, operably connected to a polynucleotide encoding a polypeptide comprising an amino acid sequence that is at least 90% or at least 95% identical to SEQ ID NO: 4 or 25. Soybean plants and seeds comprising increased protein content, which comprise the recombinant constructs are provided, wherein the polypeptide is expressed in the seed or seed produced by the plant which seed has increased protein content compared to a control seed not expressing the polypeptide.

In some embodiments, a guide RNA sequence is provided that targets a plant cell genomic locus comprises a polynucleotide that encodes a polypeptide comprising an amino acid sequence that is at least 90% or at least 95% identical to SEQ ID NO: 2 or 4. Recombinant DNA constructs that expresses the guide RNA and plants, seeds and plant cells comprising the guide RNA and/or recombinant constructs, which constructs may be stably incorporated into the genome, are provided.

In some embodiments, the DNA constructs, and plants, plant cells and seeds having the DNA constructs stably integrated into the genome, further comprise a heterologous nucleic acid sequence selected from the group consisting of: a reporter gene, a selection marker, a disease resistance gene, a herbicide resistance gene, an insect resistance gene; a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in increasing nutrient utilization efficiency, a gene involved in cold resistance, a gene involved in heat resistance and a gene involved in salt resistance in plants.

### BRIEF DESCRIPTION OF THE DRAWINGS

FIG. 1 is a schematic drawing showing the genomic map of the high-protein region on chromosome 20 and fine mapping using three deletion lines.

FIG. 2 is a sequence alignment of the partial genomic sequences for glyma.20g085100 (positions 5948 to 6497 of SEQ ID NO: 9) and its paralogue glyma.10g134400 (positions 6086 to 6312 of SEQ ID NO: 10) each from Glycine max Williams 82, and the sojasc125-pgfp01000066 paralogue from Glycine soja (positions 5951-6179 of SEQ ID NO:11).

FIG. 3 is a sequence alignment of the polypeptides glyma.20g085100 (SEQ ID NO: 2) and its paralogue glyma.10g134400 (SEQ ID NO: 6), each from Glycine max Williams 82, and the sojasc125-pgfp01000066 paralogue from Glycine soja (SEQ ID NO: 8). (Non-homologous C-terminal region of glyma.20g085100 is underlined).

FIG. 4 is a schematic drawing depicting the allele and corresponding polypeptide of glyma.20g085100 compared with the allele and corresponding polypeptide from Glycine soja.

FIG. 5 is a sequence alignment of the polynucleotides encoding glyma.20g085100 with the 321 base insertion removed and glyma.10g134400 (non-homologous residues are underlined).

FIG. 6. is a graph showing that the deletion of the 321 base pair insertion in the CCT-domain of glyma.20g085100 increases protein content in elite soybean seeds.

FIG. 7. is a graph showing the loss-of-function mutations in glyma.20g085100 increase result in an increase in protein content in elite soybean seeds.

### BRIEF DESCRIPTION OF THE SEQUENCES

TABLE 1
Listing of sequences used in this application

| Sequence Description | SEQ ID NO: |
|---|---|
| Polynucleotide encoding the glyma.20g085100 | 1 |
| CCT-domain polypeptide from Williams 82 | |
| Glyma.20g085100 CCT-domain polypeptide | 2 |
| Glyma.20g085100 polynucleotide encoding the | 3 |
| modified | |
| glyma.20g085100 CCT-domain polypeptide | |
| (insertion removed) | |
| Predicted Modified glyma.20g085100 CCT-domain | 4 |
| polypeptide (insertion removed) | |
| Polynucleotide encoding the glyma.10g134400 | 5 |
| CCT-domain polypeptide | |
| Glyma.10g134400 CCT-domain polypeptide | 6 |
| Polynucleotide encoding the sojasc125- | 7 |
| pgfp01000066 polypeptide | |
| Sojasc125-pgfp01000066 CCT-domain polypeptide | 8 |
| Glyma.20g085100 genomic polynucleotide | 9 |
| Glyma.20g085100 genomic polynucleotide | 10 |
| (insertion removed) | |
| Glyma.10g134400 genomic polynucleotide | 11 |
| Sojasc125-pgfp01000066 genomic polynucleotide | 12 |
| Guide RNA sequence GM-CCT-CR2 | 13 |
| Guide RNA sequence GM-CCT-CR3 | 14 |
| Guide RNA sequence GM-CCT-CR1 | 15 |
| Guide RNA sequence GM-CCT-CR4 | 16 |
| Guide RNA sequence GM-HP-CR40 | 17 |
| Guide RNA sequence GM-HP-CR42 | 18 |
| Guide RNA sequence GM-HP-CR41 | 19 |
| Guide RNA sequence GM-HP-CR44 | 20 |
| Guide RNA sequence GM-HP-CR43 | 21 |
| Guide RNA sequence GM-HP-CR45 | 22 |
| Polynucleotide ZM-AS2 2X repeated EME sequence | 23 |
| (modified Zea mays) | |
| Glyma.20g085100 CCT-domain polynucleotide | 24 |
| (insertion removed) - alternatively spliced | |
| Predicted Modified glyma.20g085100 CCT-domain | 25 |
| polypeptide (insertion removed) - alternatively | |
| spliced | |

### DETAILED DESCRIPTION

Compositions and methods related to modified plants producing seeds high in protein or oil are provided. Plants that have been modified using genomic editing techniques, transformation or mutagenesis to produce seeds having increased protein or increased oil are provided. Suitable plants include oil-seed plants, such as palm, canola, sunflower and soybean as well as, without limitation, rice, cotton, sorghum, wheat, maize, alfalfa and barley. Modifying expression of a CCT (CONSTANS, CO-like and TOC1) domain polypeptide in a plant such as soybean, or modifying the coding sequence of the CCT-domain polypeptide, or homologue or paralogue to produce or suppress expression of a CCT-domain polypeptide, results in a seed with altered-seed protein or oil relative to a comparable seed not comprising the modification. The modification can be introduced using genomic editing technology, transformation or mutagenesis, such as described herein. Plants, such as soybean plants, that express the modified CCT-domain polypeptide and which are robust, high-yielding and produce seeds containing increased protein or increased oil are provided. Unless specified otherwise, protein and oil and other components are measured at or adjusted to a 13% moisture basis in the soybean seed. When referring to CCT-domain polynucleotides and polypeptides herein, reference is made to both polynucleotides encoding and polypeptides containing CCT-domains, and those which would encode or contain a CCT-domain but for a nucleotide modification, such as an insertion, which disrupts the CCT-domain.

Provided are soybean seeds (and plants producing the seeds) comprising a modification and having a protein content increase in the seed of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 and less than 3.0, 2.9, 2.8, 2.7, 2.6, 2.5, 2.4, 2.3, 2.2, 2.1, 2.0, 1.9, 1.8, 1.7, 1.6, or 1.5 percentage points by weight compared with an unmodified, control, null or wild-type soybean seed (and plant producing the seed) not comprising the modification. Provided are soybean seeds having a protein content of at least 30.0%, 30.5%, 31.0%, 31.5%, 32.0%, 32.5%, 33.0%, 33.5%, 34.0%, 34.5%, 35.0%, 35.5%, 36.0%, 36.5%, 37.0%, 37.5%, 38.0%, 38.5%, 39.0%, 39.5%, 40.0%, 40.5%, 41.0%, 41.5% or 42.0% (percentage points by weight) and less than 55%, 54%, 53%, 52%, 51%, 50%, 49%, 48%, 47%, 46%, 45% or 44% (percentage points by weight).

Provided are soybean seeds (and plants producing the seeds) comprising a modification and having an oil content increase in the seed of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0% (percentage points by weight) and less than 8.0, 7.9, 7.8, 7.7, 7.6, 7.5, 7.4, 7.3, 7.2, 7.1, 7.0, 6.9, 1.8, 6.7, 6.6, 6.5, 6.4, 6.3, 6.2, 6.1, 6.0, 5.9, 5.8, 5.7, 5.6, 5.5, 5.4, 5.3, 5.2, 5.1 or 5.0% (percentage points by weight) compared with an unmodified, control, null or wild-type soybean seed (and plant producing the seed) not comprising the modification. Provided are soybean seeds having an oil content in the seeds of at least 15%, 16%, 17%, 18%, 19% or 20% (percentage points by weight) and less than about 30%, 29%, 28%, 27%, 26%, 25%, 24%, 23%, 22% or 21% (percentage points by weight).

Provided are soybean seeds (and plants producing the seeds) comprising a modification having a fiber content decrease in the seed of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4 6.0 and less than 8.0, 7.9, 7.8, 7.7, 7.6, 7.5, 7.4, 7.3, 7.2, 7.1, 7.0, 6.9, 1.8, 6.7, 6.6, 6.5, 6.4, 6.3, 6.2, 6.1, 6.0, 5.9, 5.8, 5.7, 5.6, 5.5, 5.4, 5.3, 5.2, 5.1 or 5.0 percentage points by weight compared with an unmodified, control, null or wild-type soybean seed (and plant producing the seed) not comprising the modification. Provided are soybean seeds having a fiber content in the seeds of less than 8.0, 7.5, 7.0, 6.5, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1 or 3.0% (percentage points by weight) and at least 1.0, 1.5, 2.0, 2.5 or 3.0% (percentage points by weight).

Plants are provided which contain a modification disclosed herein and which have a yield of soybean seeds by weight at 13% moisture that is at least 80%, 81%, 82%, 83%, 84%, 85%, 86%, 87%, 88%, 89%, 90%, 91%, 92%, 93%, 94%, 95%, 96%, 97%, 98% 99%, 100%, 101%, 102%, 103%, 104%, 105%, 106%, 107%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134% or 135% and less than 250%, 240%, 203%, 220%, 210%, 200%, 195%, 190%, 185%, 180%, 175%, 170%, 165%, 160%, 155%, 150%, 145% or 140% of the yield of seeds by weight of soybean variety 93B83 (U.S. Pat. No. 5,792,909), when grown under the same environmental conditions. Representative seed of soybean variety 93B83 were deposited under ATCC Accession No. 209766 on Apr. 10, 1998. As used herein, “under the same environmental conditions” means the plants are grown in proximity in the field or a greenhouse under non-stress conditions suitable for growth of a soybean plant to maturity, with the plants being exposed to the same environment and seeds harvested from each plant at maturity growth stage R8.

Applicant has made a deposit of at least 2500 seeds of Soybean Variety 93B83 with the American Type Culture Collection (ATCC), 10801 University Boulevard, Manassas, Va. 20110 USA, as ATCC Deposit No. 209766. The seeds were deposited with the ATCC on Apr. 10, 1998. This deposit of the Soybean Variety 93B83 will be maintained in the ATCC depository, which is a public depository, for a period of 30 years, or 5 years after the most recent request, or for the effective life of the patent, whichever is longer, and will be replaced if it becomes nonviable during that period. Additionally, Applicant has satisfied all the requirements of 37 C.F.R. §§ 1.801-1.809. Upon allowance of any claims in the application, the Applicant(s) will maintain and will make this deposit available to the public pursuant to the Budapest Treaty.

The soybean seeds can be efficiently processed to produce meal (either high-protein meal produced from dehulled beans or conventional meal produced from whole soybeans) having a high protein content compared with comparable meal produced from comparable seeds that do not contain the modification. In some embodiments, meal is provided which has a protein content that is increased by at least 0.5, 1.0, 1.5, 2.0, 2.5, 3.0, 3.5, 4.0, 4.5 or 5.0% percent by weight and less than 12.0, 11.0, 10.0, 9.0, 8.0, 7.0, 6.0 or 5.0% by weight compared to meal prepared from an unmodified, control, null or wild-type soybean seed not comprising the modification. The meal may be prepared from a plant seed comprising the modification and may comprise a modified polynucleotide described herein.

The modified polypeptides and polynucleotides described herein include or encode polypeptides which comprise a CCT (CONSTANS, CO-like and TOC1) domain. The CCT domain is a highly-conserved amino-acid sequence of about 43 amino acids often found in light signal transduction proteins and proteins having a role in modulating flowering time, with pleiotropic effects on morphological traits and stress tolerances in rice, maize, and other cereal crops (See, e.g., Yipu Li and Mingliang Xu, 2017, CCT family genes in cereal crops: A current overview. The Crop Journals 449-458). The function of CCT-domain protein in soybean is unknown. Unless expressly stated to the contrary, “soybean” means a soybean plant or seed of Glycine max. The CCT domain occurs at positions 326-370 in SEQ ID NO: 6 (glyma.10g134400 protein sequence); at positions 327-370 in SEQ ID NO: 4 (glyma.20g850100 protein sequence with 321 base pair (bp) insertion removed) and at positions 320-336 in SEQ ID NO: 8 (sojasc125-pgfp01000066 protein sequence from Glycine soja.

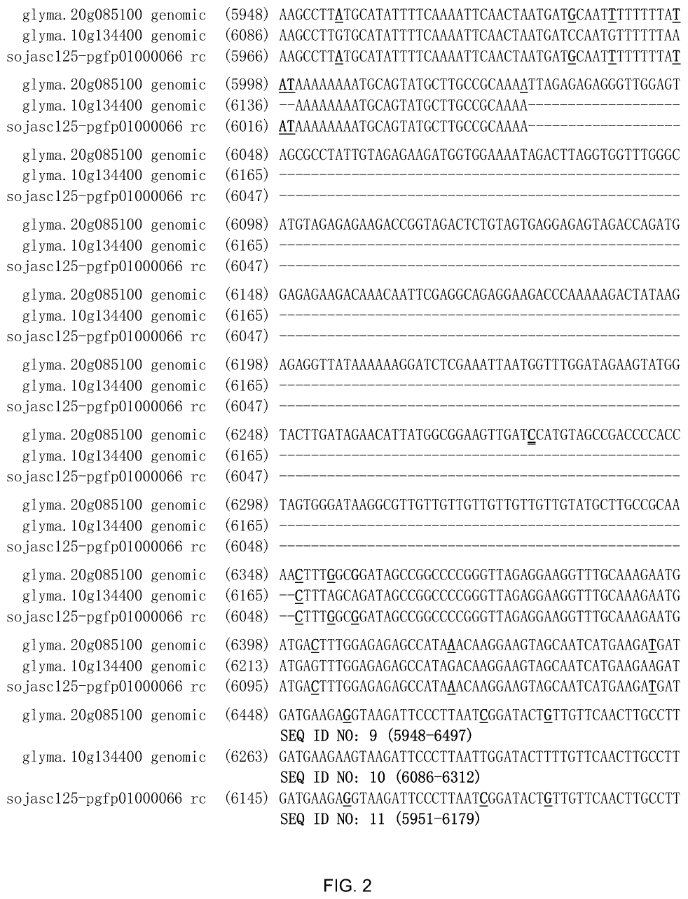

Examples of polypeptides include those encoded by two gene paralogues found in Glycine max soybean: glyma.20g085100 (SEQ ID NO: 1) a polynucleotide encoding a disrupted CCT-domain polypeptide (SEQ ID NO: 2; 85100 CCT protein) located on soybean chromosome 20 and glyma.10g134400 (SEQ ID NO: 5) located on chromosome 10 encoding a CCT-domain polypeptide (SEQ ID NO: 6). The paralogues share homology with each other at the N-terminus and with an allele found in wild soybean Glycine soja: sojasc125-pgfp01000066 (SEQ ID NO: 7) encoding the sojasc125-pgfp01000066 polypeptide (SEQ ID NO: 8). “Glyma.20g085100” is used interchangeably herein with “85100 CCT” protein, polypeptide or polynucleotide. “Glyma.10g134400” is used interchangeably herein with “134400 CCT” protein, polypeptide or polynucleotide. “Sojasc125-pgfp01000066” is used interchangeably herein with “1000066 CCT” protein, polypeptide or polynucleotide. The 85100 CCT protein is encoded by a nucleotide which includes a 321 base-pair insertion not found in the nucleotide encoding the 134400 CCT protein or the nucleotide encoding the 1000066 CCT protein, resulting in the encoding of a protein that does not contain a CCT domain. The insertion occurs from position 6029 to 6349 of SEQ ID NO: 9, corresponding to the position after 352 of SEQ ID NO: 2. However, at the 321-bp insertion site there is a 17 base pair duplication, the insertion could thus also occur at positions 6012 to 6332 of SEQ ID NO: 9. Modifications of sequences corresponding to either location may be performed. The 321 base pair (bp) insertion causes a frame-shift such that the 4-exon coding sequence, such as found in the genomic region on chromosome 10 (SEQ ID NO:10) becomes a 5-exon coding sequence on chromosome 20, and such that the C-terminal region of the 85100 CCT protein (from position 323 to 443 of SEQ ID NO: 2) is a new sequence lacking the CCT domain and different from the C-terminus of the 134400 CCT protein and the 1000066 CCT protein. FIG. 2 shows the alignment of these three polynucleotides with the non-aligned C-terminal region underlined.

In some embodiments, the modification comprises a modification on soybean chromosome 20 to delete all or part of the 321 bp insertion found in SEQ ID NO: 9 (positions 6029 to 6349 or 6012 to 6332), to produce a coding sequence such as shown in SEQ ID NO: 3, which encodes a modified 85100 CCT protein shown in SEQ ID NO: or the alternatively spliced CCT protein shown in SEQ ID NO: 25, or which encodes a polypeptide functional to increase protein and sharing a percent identity with SEQ ID NO: 4 or 25 as described herein. The polynucleotide coding sequences for SEQ ID NO: 4 and 25 are shown as SEQ ID NO: 3 and 24 respectively. In some embodiments, the deletion is 3, 6, 9 or 12 base pairs longer or shorter than the 321 bp insertion, resulting in a deletion of 309, 312, 315, 318, 321, 324, 327, 330 or 333 bp or a deletion of at least 309, 312, 315, 318, 321, 324, 327, 330 and less than 333, 330, 327, 324, 321, 318, 315, or 312 bp. The sequence containing the deletion produces a functional CCT-domain polypeptide that has one, two, three or four amino acids fewer or more at the region corresponding to the 321 bp insertion site. The deletion can begin at the position corresponding to 6003, 6006, 6009, 6012, 6015, 6018, or 6021 of SEQ ID NO: 9 and end at the position corresponding to 6323, 6326, 6329, 6332, 6335, 6338, or 6341 of SEQ ID NO: 9. The deletion can begin at the position corresponding to 6020, 6023, 6026, 6029, 6032, 6035, or 6038 of SEQ ID NO: 9 and end at the position corresponding to 6340, 6343, 6346, 6349, 6352, 6355 or 6358 of SEQ ID NO: 9. The deletion can begin at the position corresponding to 6003, 6006, 6009, 6012, 6015, 6018, or 6021 6020, 6023, 6026, 6029, 6032, 6035, or 6038 of SEQ ID NO: 9 and end at the position corresponding to 6323, 6326, 6329, 6332, 6335, 6338, 6341, 6340, 6343, 6346, 6349, 6352, 6355 or 6358 of SEQ ID NO: 9. The plants produce seeds with increased protein as described herein. The genome can be further modified to include a sequence that increases expression of the modified 85100 CCT protein as disclosed herein.

In some embodiments, the modification results in the suppression of the native glyma.20g085100 polypeptide which does not contain a CCT-domain (e.g. SEQ ID NO: 2). The genome is modified to knock-out, silence, reduce or suppress expression of the native glyma.20g085100 polypeptide, such as by disrupting the reading frame through insertion or deletion of one or more single bases or short or long sequences, introducing a sufficient number of SNPs to disrupt function or by modifying a transcription regulatory sequence in the transcription regulatory region to include for example repressor elements, repressor binding elements or disrupted promotor enhancer elements to reduce or prevent expression of the glyma.20g085100 polypeptide. In some embodiments, the expression level of the polynucleotide or polypeptide in a tissue or organ of interest, such as the seed, seed endosperm, embryo, leaf, root or stalk, is less than 95, 90, 85, 80, 75, 70, 65, 60, 55, 50, 45, 40, 35, 30, 25, 20, 15, 10, 5, 4, 3, 2, or 1% of the expression level of the polynucleotide or polypeptide in a comparable control, unmodified or null tissue or organ of interest. Plants producing seeds with increased protein as described herein are obtained.

In some embodiments, the modification comprises a modification on soybean chromosome 10 to enhance expression of a 134400 CCT protein or a modified 85100 CCT protein. The genome can be modified to insert a regulatory element such as promoter enhancing element or an element to prevent activity of a repressor of transcription such that expression of the 134400 CCT protein or modified 85100 CCT protein is increased. Transgenic plants comprising constructs containing a polynucleotide encoding a 134400 CCT polypeptide or a modified 85100 CCT protein operably connected to a heterologous regulatory element are provided. Heterologous means that the sequences are from a different location, chromosome or chromosome region in the genome of the organism, or are from different species and are not found in nature together. The plants produce seeds with increased protein as described herein.

In some embodiments, the soybean plant further includes a heterologous nucleic acid sequence selected from the group consisting of: a reporter gene, a selection marker, a disease resistance gene, a herbicide resistance gene, an insect resistance gene; a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in increasing nutrient utilization efficiency, a gene involved in cold resistance, a gene involved in heat resistance and a gene involved in salt resistance in plants.

Provided are polynucleotides that have at least about or at least 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to a reference nucleotide sequence, such as a nucleotide sequence disclosed in the sequence listing herein, using one of the alignment programs described herein using standard parameters, as well as nucleotide substitutions, deletions, insertions, fragments thereof, and combinations thereof.

An “isolated polynucleotide” generally refers to a polymer of ribonucleotides (RNA) or deoxyribonucleotides (DNA) that is single- or double-stranded, optionally containing synthetic, non-natural or altered nucleotide bases, that is no longer in its natural environment and have been placed in a difference environment by the hand of man, for example in vitro. An isolated polynucleotide in the form of DNA may be comprised of one or more segments of cDNA, genomic DNA or synthetic DNA.

A “recombinant” nucleic acid molecule (or DNA) is used herein to refer to a nucleic acid sequence (or DNA) that is in a recombinant bacterial or plant host cell. In some embodiments, an “isolated” or “recombinant” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived.

The terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

A transcription regulatory element or sequence or a regulatory element or sequence generally refers to a transcriptional regulatory element involved in regulating the transcription of a nucleic acid molecule such as a gene or a target gene. The regulatory element is a nucleic acid and may include a promoter, an enhancer, an intron, a 5′-untranslated region (5′-UTR, also known as a leader sequence), or a 3′-UTR or a combination thereof. A regulatory element may act in “cis” or “trans”, and generally it acts in “cis”, i.e. it activates expression of genes located on the same nucleic acid molecule, e.g. a chromosome, where the regulatory element is located. The nucleic acid molecule regulated by a regulatory element does not necessarily have to encode a functional peptide or polypeptide, e.g., the regulatory element can modulate the expression of a short interfering RNA or an anti-sense RNA.

In some embodiments, the modified polynucleotide includes a modified transcriptional enhancer sequence. An enhancer element is any nucleic acid molecule that increases transcription of a nucleic acid molecule when functionally linked to a promoter regardless of its relative position. An enhancer may be an innate element of the promoter or a heterologous element inserted to enhance the amount of promotor activity or tissue-specificity of a promoter.

Various enhancers may be used including introns with gene expression enhancing properties in plants (US Patent Application Publication Number 2009/0144863), the ubiquitin intron (i.e., the maize ubiquitin intron 1 (see, for example, NCBI sequence S94464)), the omega enhancer or the omega prime enhancer (Gallie, et al., (1989) Molecular Biology of RNA ed. Cech (Liss, New York) 237-256 and Gallie, et al., (1987) Gene 60:217-25), the CaMV 35S enhancer (see, e.g., Benfey, et al., (1990) EMBO J. 9:1685-96) and the enhancers of U.S. Pat. No. 7,803,992 may also be used, each of which is incorporated by reference. The above list of transcriptional enhancers is not meant to be limiting. Any appropriate transcriptional enhancer can be used in the embodiments.

A repressor (also sometimes called herein silencer, repressor element or repressor binding element) is defined as any nucleic acid molecule which inhibits the transcription when functionally linked to a promoter regardless of relative position.

“Promoter” generally refers to a nucleic acid fragment capable of controlling transcription of another nucleic acid fragment. A promoter generally includes a core promoter (also known as minimal promoter) sequence that includes a minimal regulatory region to initiate transcription, that is a transcription start site. Generally, a core promoter includes a TATA box and a GC rich region associated with a CAAT box or a CCAAT box. These elements act to bind RNA polymerase I to the promoter and assist the polymerase in locating the RNA initiation site. Some promoters may not have a TATA box or CAAT box or a CCAAT box, but instead may contain an initiator element for the transcription initiation site. A core promoter is a minimal sequence required to direct transcription initiation and generally may not include enhancers or other UTRs. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Core promoters are often modified to produce artificial, chimeric, or hybrid promoters, and can further be used in combination with other regulatory elements, such as cis-elements, 5′UTRs, enhancers, or introns, that are either heterologous to an active core promoter or combined with its own partial or complete regulatory elements.

The term “cis-element” generally refers to transcriptional regulatory element that affects or modulates expression of an operably linked transcribable polynucleotide, where the transcribable polynucleotide is present in the same DNA sequence. A cis-element may function to bind transcription factors, which are trans-acting polypeptides that regulate transcription.

The termination region may be native with the transcriptional initiation region, may be native with the operably linked DNA sequence of interest, may be native with the plant or may be derived from another source (i.e., foreign or heterologous to the promoter, the sequence of interest, the plant or any combination thereof).

The sequences include one or more contiguous nucleotides. “Contiguous nucleotides” is used herein to refer to nucleotide residues that are immediately adjacent to one another.

As used herein non-genomic nucleic acid sequence, nucleic acid molecule or polynucleotide refers to a nucleic acid molecule that has one or more changes in the nucleic acid sequence compared to a native or genomic nucleic acid sequence. In some embodiments, the change to a native or genomic nucleic acid molecule includes but is not limited to: changes in the nucleic acid sequence due to the degeneracy of the genetic code; optimization of the nucleic acid sequence for expression in plants; changes in the nucleic acid sequence to introduce at least one amino acid substitution, insertion, deletion and/or addition compared to the native or genomic sequence; deletion of one or more upstream or downstream regulatory regions associated with the genomic nucleic acid sequence; insertion of one or more heterologous upstream or downstream regulatory regions; deletion of the 5′ and/or 3′ untranslated region associated with the genomic nucleic acid sequence; insertion of a heterologous 5′ and/or 3′ untranslated region; and modification of a polyadenylation site. In some embodiments, the non-genomic nucleic acid molecule is a synthetic nucleic acid sequence.

Provided are polypeptides having at least about or at least 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to polypeptides referenced in the sequence listing, as well as amino acid substitutions, deletions, insertions, fragments thereof, and combinations thereof. The term “about” when used herein in context with percent sequence identity means +/−0.5%. These values can be appropriately adjusted to determine corresponding homology of proteins considering amino acid similarity and the like.

In some embodiments, the sequence identity is against the full-length sequence of a polypeptide disclosed in the sequence listing. In some embodiments, the polypeptide retains activity or shows enhanced or reduced activity

As used herein, the term “protein,” “peptide molecule,” or “polypeptide” includes those molecules that undergo modification, including post-translational modifications, such as, but not limited to, disulfide bond formation, glycosylation, phosphorylation or oligomerization.

The terms “amino acid” and “amino acids” refer to all naturally occurring L-amino acids.

Variants may be made by making random mutations or the variants may be designed. In the case of designed mutants, there is a high probability of generating variants with similar activity to the native polypeptide when amino acid identity is maintained in critical regions of the polypeptide which account for biological activity or are involved in the determination of three-dimensional configuration which ultimately is responsible for the biological activity. A high probability of retaining activity will also occur if substitutions are conservative. Amino acids may be placed in the following classes: non-polar, uncharged polar, basic, and acidic. Conservative substitutions whereby an amino acid of one class is replaced with another amino acid of the same type are least likely to materially alter the biological activity of the variant. Table 1 provides a listing of examples of amino acids belonging to each class.

TABLE 2
Classes of amino acids

| Class of Amino Acid | Examples of Amino Acids |
|---|---|
| Nonpolar Side Chains | Ala (A), Val (V), Leu (L), Ile (I), Pro |
| | (P), Met (M), Phe (F), Trp (W) |
| Uncharged Polar Side Chains | Gly (G), Ser (S), Thr (T), Cys (C), Tyr |
| | (Y), Asn (N), Gln (Q) |
| Acidic Side Chains | Asp (D), Glu (E) |
| Basic Side Chains | Lys (K), Arg (R), His (H) |
| Beta-branched Side Chains | Thr, Val, Ile |
| Aromatic Side Chains | Tyr, Phe, Trp, His |

Alternatively, alterations may be made to the protein sequence of many proteins at the amino or carboxy terminus without substantially affecting activity. This can include insertions, deletions or alterations introduced by modern molecular methods, such as polymerase chain reaction (PCR), including PCR amplifications that alter or extend the protein coding sequence by inclusion of amino acid encoding sequences in the oligonucleotides utilized in the PCR amplification. Alternatively, the protein sequences added can include entire protein-coding sequences, to generate protein fusions. Such fusion proteins are often used to (1) increase expression of a protein of interest (2) introduce a binding domain, enzymatic activity or epitope to facilitate either protein purification, protein detection or other experimental uses (3) target secretion or translation of a protein to a subcellular organelle, such as the periplasmic space of Gram-negative bacteria, mitochondria or chloroplasts of plants or the endoplasmic reticulum of eukaryotic cells, the latter of which often results in glycosylation of the protein.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. In another embodiment, the percent identity is calculated across the entirety of the reference sequence. The percent identity between two sequences can be determined using techniques similar to those described below, with or without allowing gaps. In calculating percent identity, typically exact matches are counted. A gap, (a position in an alignment where a residue is present in one sequence but not in the other) is regarded as a position with non-identical residues.

The determination of percent identity between two sequences can be accomplished using a mathematical algorithm. A non-limiting example of a mathematical algorithm utilized for the comparison of two sequences is the algorithm incorporated into the BLASTN and BLASTX programs. Karlin and Altschul (1990) Proc. Nat'l. Acad. Sci. USA 87:2264, Altschul et al. (1990) J. Mol. Bioi. 215:403, and Karlin and Altschul (1993) Proc. Nat'l. Acad. Sci. USA 90:5873-5877. BLAST nucleotide searches can be performed with the BLASTN program, score=100, word length=12, to obtain nucleotide sequences homologous to nucleic acid molecules disclosed herein. BLAST protein searches can be performed with the BLASTX program, score=50, word length=3, to obtain amino acid sequences homologous to polypeptides disclosed herein. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection.

Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, Calif.). After alignment of amino acid sequences with ClustalW, the percent amino acid identity can be assessed. A non-limiting example of a software program useful for analysis of ClustalW alignments is GENEDOC™. GENEDOC™ (Karl Nicholas) allows assessment of amino acid (or DNA) similarity and identity between multiple proteins. Another non-limiting example of a mathematical algorithm utilized for the comparison of sequences is the algorithm of Myers and Miller (1988) CAB/OS 4(1):11-17. Such an algorithm is incorporated into the ALIGN program (version 2.0), which is part of the GCG Wisconsin Genetics Software Package, Version 10 (available from Accelrys, Inc., San Diego, Calif., USA). When utilizing the ALIGN program for comparing amino acid sequences, a PAM120 weight residue table, a gap length penalty of 12, and a gap penalty of 4 can be used. Unless otherwise stated, GAP Version 10, which uses the algorithm of Needleman and Wunsch (1970) J. Mol. Biol. 48(3):443-453, will be used to determine sequence identity or similarity using the following parameters: % identity and % similarity for a nucleotide sequence using GAP Weight of 50 and Length Weight of 3, and the nwsgapdna.cmp scoring matrix; % identity or % similarity for an amino acid sequence using GAP weight of 8 and length weight of 2, and the BLOSUM62 scoring program. Equivalent programs may also be used. By “equivalent program” is intended any sequence comparison program that, for any two sequences in question, generates an alignment having identical nucleotide residue matches and an identical percent sequence identity when compared to the corresponding alignment generated by GAP Version 10.

Isolated or recombinant nucleic acid molecules comprising nucleic acid sequences encoding CCT-domain polypeptides or biologically active portions thereof, as well as nucleic acid molecules sufficient for use as hybridization probes to identify nucleic acid molecules encoding proteins with regions of sequence homology are provided. As used herein, the term “nucleic acid molecule” refers to DNA molecules (e.g., recombinant DNA, cDNA, genomic DNA, plastid DNA, mitochondrial DNA) and RNA molecules (e.g., mRNA) and analogs of the DNA or RNA generated using nucleotide analogs. The nucleic acid molecule can be single-stranded or double-stranded, but preferably is double-stranded DNA.

Nucleotide sequences that encode CCT-domain polypeptides, variants and truncations, may be synthesized and cloned into standard plasmid vectors by conventional means, or may be obtained by standard molecular biology manipulation of other constructs containing the nucleotide sequences.

In some embodiments, the nucleic acid molecule encoding a CCT-domain polypeptide is a polynucleotide having the sequence set forth in SEQ ID NO: 1, 3, 5, 7, 9, 10, 11 or 12 and variants, fragments and complements thereof. Nucleic acid sequences that are complementary to a nucleic acid sequence of the embodiments or that hybridize to a sequence of the embodiments are also encompassed. The nucleic acid sequences can be used in DNA constructs or expression cassettes for transformation and expression in organisms, including microorganisms and plants. The nucleotide or amino acid sequences may be synthetic sequences that have been designed for expression in an organism including, but not limited to, a microorganism or a plant.

In some embodiments, the nucleic acid molecule encoding the polypeptide is a non-genomic nucleic acid sequence.

In some embodiments, the nucleic acid molecule encoding a polypeptide is a non-genomic polynucleotide having a nucleotide sequence having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity, to the nucleic acid sequence of SEQ ID NO: 1, 3, 5 or 7 wherein the encoded polypeptide is functional to increase protein in a soybean seed.

In some embodiments, the polynucleotide encodes a polypeptide having, or the polypeptide has, at least about 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 2, 4, 6 or 8 and optionally has at least one amino acid substitution, deletion, insertion or combination therefore, compared to the native sequence.

In some embodiments, the nucleic acid molecule encodes a polypeptide comprising, or the polypeptide comprises, an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater identity across the entire length of the amino acid sequence of SEQ ID NO: 2, 4, 6 or 8.

In some embodiments, the nucleic acid encodes a polypeptide having, or the polypeptide has, at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or greater sequence identity compared to SEQ ID NO: 2, 4, 6 or 8. In some embodiments, the sequence identity is calculated using ClustalW algorithm in the ALIGNX® module of the Vector NTI® Program Suite (Invitrogen Corporation, Carlsbad, Calif.) with all default parameters. In some embodiments, the sequence identity is across the entire length of polypeptide calculated using ClustalW algorithm in the ALIGNX module of the Vector NTI Program Suite (Invitrogen Corporation, Carlsbad, Calif.) with all default parameters.

The embodiments also encompass nucleic acid molecules encoding COT-domain polypeptide variants. “Variants” of the polypeptide encoding nucleic acid sequences include those sequences that encode the polypeptides disclosed herein but that differ conservatively because of the degeneracy of the genetic code as well as those that are sufficiently identical as discussed above. Naturally occurring allelic variants can be identified with the use of well-known molecular biology techniques, such as polymerase chain reaction (PCR) and hybridization techniques as outlined below. Variant nucleic acid sequences also include synthetically derived nucleic acid sequences that have been generated, for example, by using site-directed mutagenesis but which still encode the polypeptides disclosed as discussed below.

Oligonucleotide probes and methods for detecting the polynucleotides described herein are provided. Oligonucleotide probes are detectable nucleotide sequences, such as by an appropriate radioactive label or may be fluorescence as described in, for example, U.S. Pat. No. 6,268,132. As is well known in the art, if the probe molecule and nucleic acid sample hybridize by forming strong base-pairing bonds between the two molecules, it can be reasonably assumed that the probe and sample have substantial sequence homology. Preferably, hybridization is conducted under stringent conditions by techniques well-known in the art, as described, for example, in Keller and Manak (1993). Detection of the probe provides a means for determining in a known manner whether hybridization has occurred. Such a probe analysis provides a rapid method for identifying modified genes of CCT-domain polypeptides, which modified genes and methods are provided. The nucleotide segments which are used as probes can be synthesized using a DNA synthesizer and standard procedures. These nucleotide sequences can also be used as PCR primers to amplify genes.

As is well known to those skilled in molecular biology, similarity of two nucleic acids can be characterized by their tendency to hybridize. Provided are nucleic acids that hybridize to those sequences disclosed herein under stringent conditions. As used herein the terms “stringent conditions” or “stringent hybridization conditions” are intended to refer to conditions under which a probe or nucleic acid will hybridize (anneal) to a particular sequence to a detectably greater degree than to other sequences (e.g. at least 2-fold over background).

Provided are nucleotide constructs comprising sequences described herein. The use of the term “nucleotide constructs” herein is not intended to limit the embodiments to nucleotide constructs comprising DNA. Nucleotide constructs particularly polynucleotides and oligonucleotides composed of ribonucleotides and combinations of ribonucleotides and deoxyribonucleotides may also be employed in the methods disclosed herein. The nucleotide constructs, nucleic acids, and nucleotide sequences of the embodiments additionally encompass all complementary forms of such constructs, molecules, and sequences. Further, the nucleotide constructs, nucleotide molecules, and nucleotide sequences of the embodiments encompass all nucleotide constructs, molecules, and sequences which can be employed in the methods of the embodiments for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs, nucleic acids, and nucleotide sequences of the embodiments also encompass all forms of nucleotide constructs including, but not limited to, single-stranded forms, double-stranded forms, hairpins, stem-and-loop structures and the like.

Provided are plants, plant cells, plant seeds and plant nuclei that are modified by gene editing. In some embodiments, gene editing may be facilitated through the induction of a double-stranded break (DSB) or single-strand break, in a defined position in the genome near the desired alteration. DSBs can be induced using any DSB-inducing agent available, including, but not limited to, TALENs (transcription activator-like effector nucleases), meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template. In some embodiments, the methods do not use TALENs enzymes or technology and plants and seeds are produced from methods which do not use TALENs enzymes or technology.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease. Tethered DNAs can allow for co-localizing target and template DNA, useful in genome editing and targeted genome regulation, and can also be useful in targeting post-mitotic cells where function of endogenous HR machinery is expected to be highly diminished (Mali et al. 2013 Nature Methods Vol. 10: 957-963.) The polynucleotide modification template may be present transiently in the cell or it can be introduced via a viral replicon.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

TAL effector nucleases (TALEN) are a class of sequence-specific nucleases that can be used to make double-strand breaks at specific target sequences in the genome of a plant or other organism. (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered.

Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, WO2016007347, published on Jan. 14, 2016, and WO201625131, published on Feb. 18, 2016, all of which are incorporated by reference herein.

The term “Cas gene” herein refers to a gene that is generally coupled, associated or close to, or in the vicinity of flanking CRISPR loci in bacterial systems. The terms “Cas gene”, “CRISPR-associated (Cas) gene” are used interchangeably herein. The term “Cas endonuclease” herein refers to a protein encoded by a Cas gene. A Cas endonuclease herein, when in complex with a suitable polynucleotide component, is capable of recognizing, binding to, and optionally nicking or cleaving all or part of a specific DNA target sequence. A Cas endonuclease described herein comprises one or more nuclease domains. Cas endonucleases of the disclosure includes those having a HNH or HNH-like nuclease domain and/or a RuvC or RuvC-like nuclease domain. A Cas endonuclease of the disclosure includes a Cas9 protein, a Cpf1 protein, a C2c1 protein, a C2c2 protein, a C2c3 protein, Cas3, Cas 5, Cas7, Cas8, Cas10, or complexes of these.

As used herein, the terms “guide polynucleotide/Cas endonuclease complex”, “guide polynucleotide/Cas endonuclease system”, “guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component. (See also U.S. Patent Application US 2015-0082478 A1, published on Mar. 19, 2015 and US 2015-0059010 A1, published on Feb. 26, 2015, both are hereby incorporated in its entirety by reference).

A guide polynucleotide/Cas endonuclease complex can cleave one or both strands of a DNA target sequence. A guide polynucleotide/Cas endonuclease complex that can cleave both strands of a DNA target sequence typically comprise a Cas protein that has all of its endonuclease domains in a functional state (e.g., wild type endonuclease domains or variants thereof retaining some or all activity in each endonuclease domain). Non-limiting examples of Cas9 nickases suitable for use herein are disclosed in U.S. Patent Appl. Publ. No. 2014/0189896, which is incorporated herein by reference.

Other Cas endonuclease systems have been described in PCT patent applications PCT/US16/32073, filed May 12, 2016 and PCT/US16/32028 filed May 12, 2016, both applications incorporated herein by reference.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. Cas9 protein comprises a RuvC nuclease domain and an HNH (H-N-H) nuclease domain, each of which can cleave a single DNA strand at a target sequence (the concerted action of both domains leads to DNA double-strand cleavage, whereas activity of one domain leads to a nick). In general, the RuvC domain comprises subdomains I, and III, where domain I is located near the N-terminus of Cas9 and subdomains II and III are located in the middle of the protein, flanking the HNH domain (Hsu et al, Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

Any guided endonuclease can be used in the methods disclosed herein. Such endonucleases include, but are not limited to Cas9 and Cpf1 endonucleases. Many endonucleases have been described to date that can recognize specific PAM sequences (see for example—Jinek et al. (2012) Science 337 p 816-821, PCT patent applications PCT/US16/32073, filed May 12, 2016 and PCT/US16/32028 filed May 12, 2016 and Zetsche B et al. 2015. Cell 163, 1013) and cleave the target DNA at a specific position. It is understood that based on the methods and embodiments described herein utilizing a guided Cas system one can now tailor these methods such that they can utilize any guided endonuclease system.

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. The single guide polynucleotide being comprised of sequences from the crNucleotide and the tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double strand break) the target site. (See also U.S. Patent Application US 2015-0082478 A1, published on Mar. 19, 2015 and US 2015-0059010 A1, published on Feb. 26, 2015, both are hereby incorporated in its entirety by reference.)

The term “variable targeting domain” or “VT domain” is used interchangeably herein and includes a nucleotide sequence that can hybridize (is complementary) to one strand (nucleotide sequence) of a double strand DNA target site. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type Cas endonuclease, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site.

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site. A guide RNA/Cas endonuclease complex herein can comprise Cas protein(s) and suitable RNA component(s) of any of the four known CRISPR systems (Horvath and Barrangou, 2010, Science 327:167-170) such as a type I, II, or III CRISPR system. A guide RNA/Cas endonuclease complex can comprise a Type II Cas9 endonuclease and at least one RNA component (e.g., a crRNA and tracrRNA, or a gRNA). (See also U.S. Patent Application US 2015-0082478 A1, published on Mar. 19, 2015 and US 2015-0059010 A1, published on Feb. 26, 2015, both are hereby incorporated in its entirety by reference).

The guide polynucleotide can be introduced into a cell transiently, as single stranded polynucleotide or a double stranded polynucleotide, using any method known in the art such as, but not limited to, particle bombardment, Agrobacterium transformation or topical applications. The guide polynucleotide can also be introduced indirectly into a cell by introducing a recombinant DNA molecule (via methods such as, but not limited to, particle bombardment or Agrobacterium transformation) comprising a heterologous nucleic acid fragment encoding a guide polynucleotide, operably linked to a specific promoter that is capable of transcribing the guide RNA in said cell. The specific promoter can be, but is not limited to, a RNA polymerase III promoter, which allow for transcription of RNA with precisely defined, unmodified, 5′- and 3′-ends (DiCarlo et al., Nucleic Acids Res. 41: 4336-4343; Ma et al., Mol. Ther. Nucleic Acids 3:e161) as described in WO2016025131, published on Feb. 18, 2016, incorporated herein in its entirety by reference.

Provided are plants, plant cells, plant seeds and plant nuclei that are transformed with sequences described herein. Transformation may be stable or transient. “Stable transformation” as used herein means that the nucleotide construct introduced into a plant integrates into the genome of the plant and is capable of being inherited by the progeny thereof. “Transient transformation” as used herein means that a polynucleotide is introduced into the plant and does not integrate into the genome of the plant or a polypeptide is introduced into a plant. “Plant” as used herein refers to whole plants, plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells and pollen).

Transformation methods include introduction of a recombinant DNA construct comprising an expression cassette. Provided are constructs which include one or more heterologous promoter sequences operably connected to one or more polynucleotides encoding polypeptides disclosed herein and appropriate transcription termination sequences and plants, seeds, cells and nuclei containing the recombinant DNA construct or expression cassette.

Transformation methods include introduction of a suppression DNA construct or a construct that results in increased expression of a target gene, such as encoding the CCT-domain polypeptide. “Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The term “suppression” includes lower, reduce, decline, decrease, inhibit, eliminate and prevent. “Silencing” or “gene silencing” does not specify mechanism and is inclusive, and not limited to, anti-sense, cosuppression, viral-suppression, hairpin suppression, stem-loop suppression, RNAi-based approaches and small RNA-based approaches.

The embodiments further relate to plant-propagating material of a transformed plant of the embodiments including, but not limited to, seeds, tubers, corms, bulbs, leaves and cuttings of roots and shoots. Methods of plant breeding by crossing a modified plant described herein with a second different plant are provided. Progeny plants, plant cells, seeds and plant nuclei from such breeding methods are provided, such as F1 progeny plants, plant cells, seeds and plant nuclei.

Transformation of any plant species can be carried out, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables ornamentals, and conifers.

Plants of interest include grain plants that provide seeds of interest, oil-seed plants, and leguminous plants. Seeds of interest include grain seeds, such as corn, wheat, barley, rice, sorghum, rye, millet, etc. Oil-seed plants include cotton, soybean, safflower, sunflower, Brassica, maize, alfalfa, palm, coconut, flax, castor, olive, etc. Leguminous plants include beans and peas. Beans include guar, locust bean, fenugreek, soybean, garden beans, cowpea, mung bean, lima bean, fava bean, lentils, chickpea, etc.

The methods comprise providing a plant or plant cell expressing a polynucleotide encoding the polypeptide sequence disclosed herein and growing the plant or a seed thereof in a field. In some embodiments, the expression of the modified polypeptide results in a plant producing increased yield or biomass, increased seed protein, increased seed oil, or any combination thereof.

The foregoing invention has been described in detail by way of illustration and example for purposes of clarity and understanding. As is readily apparent to one skilled in the art, the foregoing disclosures are only some of the methods and compositions that illustrate the embodiments of the foregoing invention. It will be apparent to those of ordinary skill in the art that variations, changes, modifications, and alterations may be applied to the compositions and/or methods described herein without departing from the true spirit, concept, and scope of the invention.

All publications, patents, and patent applications mentioned in the specification are incorporated by reference herein for the purpose cited to the same extent as if each was specifically and individually indicated to be incorporated by reference herein.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth. Unless expressly stated to the contrary, “or” is used as an inclusive term. For example, a condition A or B is satisfied by any one of the following: A is true (or present) and B is false (or not present), A is false (or not present) and B is true (or present), and both A and B are true (or present).

The following examples illustrate particular aspects of the disclosure and are not intended in any way to limit the disclosure.

### EXAMPLES

### Example 1. Fine Mapping of a Soybean High Protein QTL

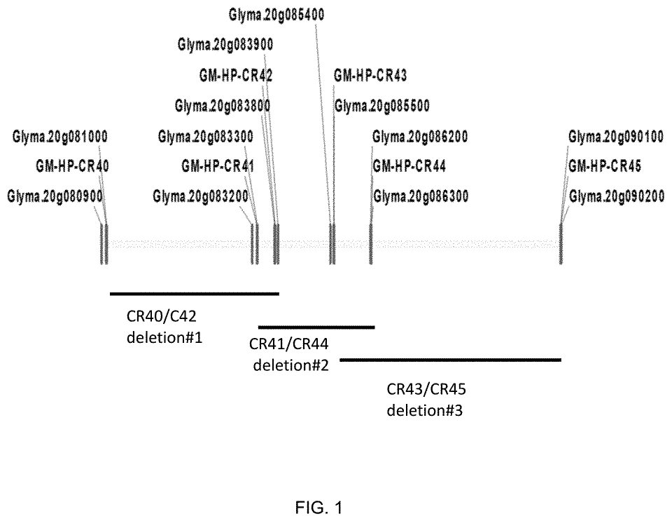

A major high protein QTL on chromosome 20 (CCT-Domain region) detected by multiple mapping studies (Chung et al 2003 Crop Sci 43:1053-1067; Nichols et al 2006 Crop Sci 46:834-839; Bolon et al. 2010 BMC Plant Biology 10:41; Hwang et al 2014 BMC genomics 15:1) was investigated. The high-protein region was mapped to a 2.4 Mb interval and could not be advanced further because of low recombination rate in the region. Using CRISPR/cas9 technology, a series of overlapping deletion regions were designed and lines are created to fine map the high-protein region (FIG. 1). The guide RNA pairs targeting specific sites within the high-protein region were designed to create overlapping dropouts in the high-protein QTL region and soybean lines were transformed. When delivered to the high-protein donor line in combination with Cas9, these guides produced and are expected to produce genomic deletions ranging from approximately 700 kb to 1.4 Mbp (Table 3).

TABLE 3
guide RNA designed to produce deletions in
CCT-Domain region of Chromosome 20

| | Approximate | | | | |
|---|---|---|---|---|---|
| Edit | expecte | | | | |
| designation | deletion | Guide 1 | Guide 1 | Guide 2 | Guide 2 |
| (guide pair) | size (bp) | name | sequence | name | sequence |
| GM-HP- | 1,041,115 | GM-HP- | GGGTATTG | GM-HP- | GGCAGTTTGG |
| CR40+42 | | CR40 (SEQ | TATGGACC | CR42 (SEQ | GATAACCCGA |
| | | ID NO: 17) | AGCA | ID NO: 18) | |
| GM-HP- | 706,332 | GM-HP- | GATGTCAT | GM-HP- | GTGGATCCAG |
| CR41+44 | | CR41 (SEQ | GAGAACTA | CR44 (SEQ | TTCACTTACT |
| | | ID NO: 19) | CGCA | ID NO: 20) | |
| GM-HP- | 1,401,600 | GM-HP- | GGCATAAG | GM-HP- | GACGCACAAT |
| CR43+45 | | CR43 (SEQ | GGCCACC | CR45 (SEQ | AACCTGACCC |
| | | ID NO: 21) | GGTGA | ID NO: 22) | |

T0 plants with deletion are selected and genotyped to verify the occurrence of the expected deletion. T0 plants may be edited on a single or both chromosomes, thus respectively hemizygous or homozygous at the edited locus. Phenotype analyses, such as protein and oil content in seeds are performed at the T1 seeds to identify the sub-region of interest that can change seed protein content. By the same mapping techniques as traditional QTL mapping using near isogeneic lines, the QTL can be mapped by overlapping deletion lines created by CRISPR/Cas9. Table 4 lists predicted protein phenotypes of deletion lines and the position of QTL. For example, if both CR40/CR42 and CR41/Cr44 deletion lines show reduced protein content while CR43/CR45 deletion line shows no protein change, the high-protein region will be defined to an interval between CR41 and CR42. An additional round of guide RNAs may be designed to further narrow down the candidate genes in the sub-region. After a candidate gene is identified, the function of the gene can be confirmed by additional editing experiments such as frame-shift knockout (silencing) or precise segment dropout and replacement.

TABLE 4
Fine mapping of high protein region on chromosome 20 based
on protein phenotype of the overlapping deletion lines

| | CR40/CR42 | CR41/CR44 | CR43/CR45 | Location of |
|---|---|---|---|---|
| | deletion | deletion | deletion | qHP20 |
| Seed protein | reduced | no change | no change | between CR40 |
| content | | | | and CR41 |
| Seed protein | reduced | reduced | no change | between CR41 |
| content | | | | and CR42 |
| Seed protein | no change | reduced | no change | between CR42 |
| content | | | | and CR43 |
| Seed protein | no change | reduced | reduced | between CR43 |
| content | | | | and CR44 |
| Seed protein | no change | no change | reduced | between CR44 |
| content | | | | and CR45 |

### Example 2. Restoration of CCT-Domain Protein to Wild Glycine soja Sequence Results in High Protein in Elite Soybeans

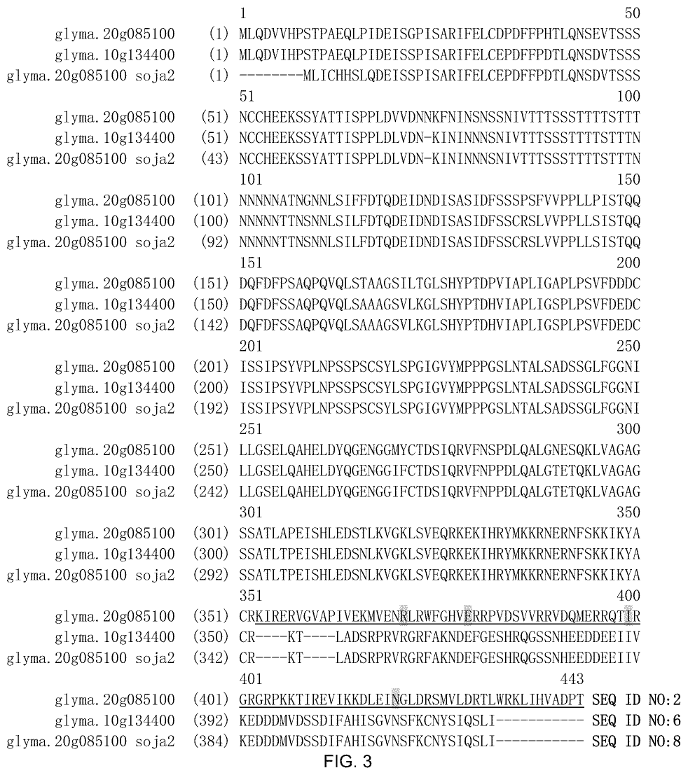

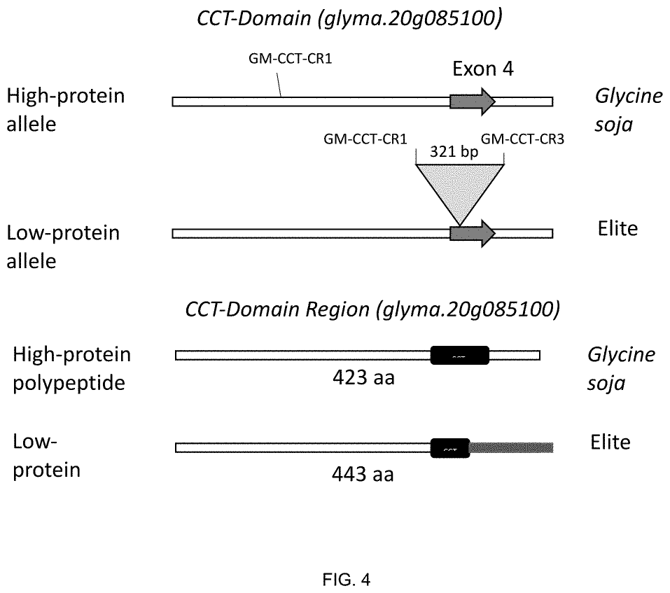

From genome sequence analysis of high-protein lines and low-protein lines, such as carried out in Example 1, one candidate gene, glyma.20g085100 was identified as a potential causative gene for high protein phenotype in the qHP20 region. Compared to high protein Glycine soja genomic sequences and soybean paralogue glyma.10g134400 found on chromosome 10, glyma.20g085100 from elite low-protein Williams82 and 93Y21 contains a 321 bp insertion in the exon 4 (FIG. 3). This insertion was identified as the potential causative mutation for the loss of high protein phenotype in the elite soybean. The 321 bp insertion was noted to be found in all elite low-protein lines but not in high-protein Danbaekkong and Glycine soja lines. Glyma.20g085100 encodes a CCT-(Constans, Co-like, and TOC1) domain protein. The 321 bp insert fragment occurs within the CCT-domain and generates a new open reading frame which produces a different 88 amino acid C-terminal sequence in the glyma.20g085100 polypeptide compared with the polypeptides encoded by the Glycine soja and glyma.10g134400 paralogues (FIG. 3; the non-identical C-terminal region of glyma.20g085100 is underlined). The disruption of CCT-domain within the protein may be responsible for the low protein content in elite soybean. FIG. 4 is a schematic showing the location of the insertion and the differences in the amino acid sequence between the Glycine soja and glyma.20g085100 paralogues.

For genome engineering applications, the type II CRISPR/Cas system minimally requires the Cas9 protein and a duplexed crRNA/tracrRNA molecule or a synthetically fused crRNA and tracrRNA (guide RNA) molecule for DNA target site recognition and cleavage (Gasiunas et al. (2012) Proc. Natl. Acad. Sci. USA 109: E2579-86, Jinek et al. (2012) Science 337:816-21, Mali et al. (2013) Science 339:823-26, and Cong et al. (2013) Science 339:819-23). Described herein is a guide RNA/Cas endonuclease system that is based on the type II CRISPR/Cas system and consists of a Cas endonuclease and a guide RNA (or duplexed crRNA and tracrRNA) that together can form a complex that recognizes a genomic target site in a plant and introduces a double-strand-break into said target site.

To use the guide RNA/Cas endonuclease system in soybean, the Cas9 gene from Streptococcus pyogenes M1 GAS (SF370) was soybean codon optimized per standard techniques known in the art. To facilitate nuclear localization of the Cas9 protein in soybean cells, Simian virus 40 (SV40) monopartite amino terminal nuclear localization signal (MAPKKKRKV) and Agrobacterium tumefaciens bipartite VirD2 T-DNA border endonuclease carboxyl terminal nuclear localization signal (KRPRDRHDGELGGRKRAR) were incorporated at the amino and carboxyl-termini of the Cas9 open reading frame, respectively. The soybean optimized Cas9 gene was operably linked to a soybean constitutive promoter such as the strong soybean constitutive promoter GM-EF1A2 (US patent application 20090133159) or regulated promoter by standard molecular biological techniques.

The second component of a functional guide RNA/Cas endonuclease system for genome engineering applications is a duplex of the crRNA and tracrRNA molecules or a synthetic fusing of the crRNA and tracrRNA molecules, a guide RNA. To confer efficient guide RNA expression (or expression of the duplexed crRNA and tracrRNA) in soybean, the soybean U6 polymerase III promoter and U6 polymerase III terminator are used.

Plant U6 RNA polymerase III promoters have been cloned and characterized from species such as Arabidopsis and Medicago truncatula (Waibel and Filipowicz, NAR 18:3451-3458 (1990); Li et al., J. Integrat. Plant Biol. 49:222-229 (2007); Kim and Nam, Plant Mol. Biol. Rep. 31:581-593 (2013); Wang et al., RNA 14:903-913 (2008)). Soybean U6 small nuclear RNA (snRNA) genes were identified by searching public soybean variety Williams82 genomic sequence using Arabidopsis U6 gene coding sequence. Approximately 0.5 kb genomic DNA sequence upstream of the first G nucleotide of a U6 gene was selected to be used as a RNA polymerase III promoter for example, GM-U6-13.1 promoter or GM-U6-9.1 promoter, to express guide RNA to direct Cas9 nuclease to designated genomic site. The guide RNA coding sequence was 76 bp long and comprised a 20 bp variable targeting domain from a chosen soybean genomic target site on the 5′ end and a tract of 4 or more T residues as a transcription terminator on the 3′ end. The first nucleotide of the 20 bp variable targeting domain was a G residue to be used by RNA polymerase III for transcription. Other soybean U6 homologous genes promoters were similarly cloned and used for small RNA expression.

Since the Cas9 endonuclease and the guide RNA need to form a protein/RNA complex to mediate site-specific DNA double strand cleavage, the Cas9 endonuclease and guide RNA are expressed in same cells. To improve their co-expression and presence, the Cas9 endonuclease and guide RNA expression cassettes are linked into a single DNA construct.

To validate the insertion as the causative mutation for low protein, a pair of guide RNA GM-CCT-CR2 and CR3 were designed to delete the insertion in elite soybean (Table 5).

TABLE 5
Example of guide RNA designed to produce modifications in
CCT-domain regions of soybean chromosomes 10 and 20

| | Approximate | | | | |
|---|---|---|---|---|---|
| Edit | expected | | | | |
| designation | deletion | Guide 1 | Guide 1 | Guide 2 | Guide 2 |
| (guide pair) | size (bp) | name | sequence | name | sequence |
| GM-CCT- | 321 | GM-CCT- | GTGCCG | GM-CCT- | GTATGCT |
| CR2+3 | | CR2 (SEQ | CAAAATT | CR3 (SEQ | TGCCGCA |
| | | ID NO: 12) | AGAGAGA | ID NO: 13) | AAACTT |

The soybean U6 small nuclear RNA promoter, GM-U6-13.1 or GM-U6-9.1 promoter was used to express guide RNAs to direct Cas9 nuclease to designated genomic target sites. A soybean codon optimized Cas9 endonuclease expression cassette and guide RNA expression cassettes were linked in the plasmid (RV029969 or RV029968). For example, the RV029969 construct, which contains the GM-CCT-CR2 and GM-CCT-CR3 gRNA expression cassettes and the Cas9 expression cassette, was made with an aim of targeting the 321 bp insertion region to restore the function of the CCT-domain protein. The second RV029968 construct, which contains the GM-CCT-CR1 gRNA expression cassette and Cas9 expression cassette, was made with an aim to knockout or silence the glyma.20g085100 CCT gene in elite and high protein lines. In the elite line, silencing the native glyma.20g085100 restored high protein phenotype. Introduction of this GM-CCT-CR1 gRNA with CAS9 into a high protein line which does not contain the 321 bp insertion prevented elevated protein content in seeds. A third RV030124 construct, which contains the GM-CCT-CR4 gRNA expression cassette and Cas9 expression cassette, will be made with an aim to knockout or silence the glyma.10g134400 gene in both elite and high protein lines. Introduction of this GM-CCT-CR4 gRNA with CAS9 into both elite and high protein line is expected to alter (increase or decrease) protein and oil content in seeds. The constructs were transformed into Ochrobactrum haywardense H1-8 strain for soybean transformation.

Ochrobactrum-mediated soybean embryonic axis transformation was done essentially as described in US Patent application publication US 2018/0216123. Mature dry seeds of soybean cultivar 93Y21 were disinfected using chlorine gas and imbibed on semi-solid medium containing 5 g/l sucrose and 6 g/l agar at room temperature in the dark. After an overnight incubation, the seeds were soaked in distilled water for an additional 3-4 hrs at room temperature in the dark. Intact embryonic axes were isolated from cotyledon using a scalpel blade in distilled sterile water. The embryonic-axis explants were transferred to the deep plate with 15 mL of Ochrobactrum haywardense H1-8 further containing a helper vector PHP85634 (RV005393) with binary vector RV029968 or RV029969 with suspension at OD600=0.5 in infection medium containing 200 μM acetosyringone. The plates were sealed with parafilm (“Parafilm M” VWR Cat #52858), then sonicated (Sonicator-VWR model 50T) for 30 seconds. After sonication, embryonic-axis explants were transferred to a single layer of autoclaved sterile filter paper (VWR #415/Catalog #28320-020). The plates were sealed with Micropore tape (Catalog #1530-0, 3M, St. Paul, Minn.)) and incubated under dim light (5-10 μE/m²/s, cool white fluorescent lamps) for 16 hrs at 21° C. for 3 days.

After co-cultivation, the embryonic-axis explants were cultured on shoot induction medium solidified with 0.7% agar in the absence of selection. The base of the explant (i.e., root radical of embryonic axis) was embedded in the medium. Shoot induction was carried out in a Percival Biological Incubator at 26° C. with a photoperiod of 18 hrs and a light intensity of 40-70 μE/m²/s. 6 to 7 weeks after transformation, elongated shoots (>1-2 cm) were isolated and transferred to rooting medium containing selection agent. Transgenic plantlets were transferred to soil pots and grown in the greenhouse.

Genomic DNA was extracted from leaf samples and analyzed by regular PCR. PCR primers were designed to amplify the genomic region of interests. The PCR bands were cloned into pCR2.1 vector using a TOPO-TA cloning kit (Invitrogen) and multiple clones were sequenced to check for target site sequence changes as the results of NHEJ. The 321 base pair dropout variants by the GM-CCT-CR2/GM-CCT-CR3 pair were identified, as well as the frameshift silenced variants by the GM-CCT-CR1 and GM-CCT-CR4. Screening of seed from edited events are performed using non-destructive single-seed near-infrared analysis (SS-NIR) to evaluate protein content and other seed components, such as oil and moisture, such as described in Example 2. Seeds containing the modifications and having high protein were identified and selected for further use.

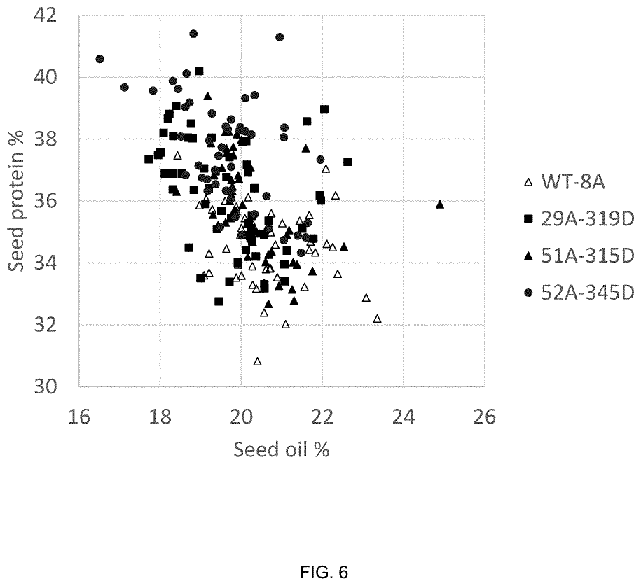

Three edited variants with 315 bp, 319 bp or 345 bp deletion were obtained in the elite soybean line 93Y21. Although the deletions were not a perfect deletion of 321 bp, a portion of T1 segregating seeds from the variants 29A-319D, 51A-315D and 52A-345D showed high protein phenotypes compared to wild type seeds, validating that the 321 bp insertion caused low protein in elite 93Y21 (FIG. 6). The results demonstrate that modification of 321 bp region increases seed protein content in elite soybean.

### Example 3: Generation of Plants Having High Protein or High Oil Through Suppression of Native Coding Sequences Provides High Protein or High Oil Seeds

To produce plants producing seeds with modified oil and protein composition, genetic modification of the native sequences in elite soy lines was carried out. A single guide RNA GM-CCT CR1 was designed to target the exon 2 of the glyma.20g085100 to knockout or silence the gene function on chromosome 20 (Table 6). Similarly, a single guide RNA GM-CCT CR4 was designed to target the exon 2 of the glyma.10g134400 to knockout or silence the glyma.10g134400 gene function (Table 6). Guide expression cassettes and transformation were carried out according to Example 2.

TABLE 6
Examples of guide RNA designed to produce
modifications in CCT domain regions
of soybean chromosomes 10 and 20

| Guide 1 name | Guide 1 sequence |
|---|---|
| GM-CCT-CR1 (SEQ ID NO: 14) | GGCACCTGTGGCTGAGCTGA |
| GM-CCT-CR4 (SEQ ID NO: 15) | GAGTGTCAAAGAGGATGGAC |

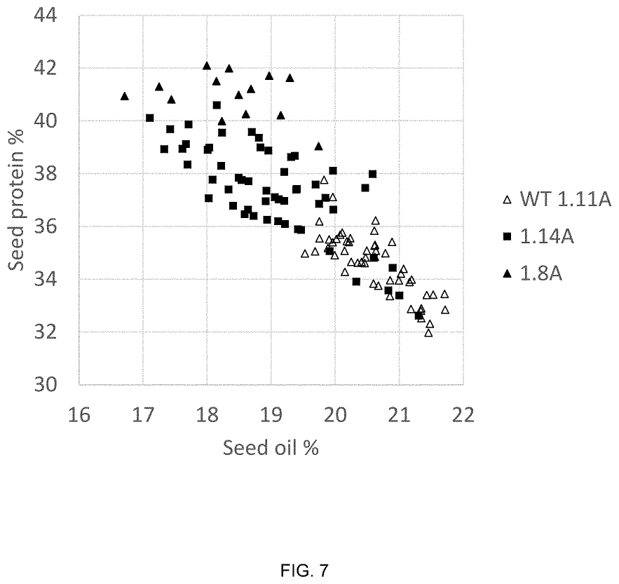

Introduction of the guide RNA (gRNA) GM-CCT CR1 with CAS9 created a frame shift mutation in the glyma.20g085100 gene. Two frame shift variants were obtained. Variant 1.8A contained a 7 bp deletion at Gm-CCT-CR1 cutting site at both alleles. T1 seeds were fixed homozygous and showed an increased seed protein content compared to wild type seeds (FIG. 7). Variant 1.14A contained a 19 bp deletion at Gm-CCT-CR1 cutting site at one allele. T1 seeds were segregating for the mutation. Compared to wild type seeds, a portion of variant 1.14A T1 seeds were high protein as shown in FIG. 7. The results show that frame shift mutations in glyma.20g085100 increased seed protein content in elite soybean. Other mutations which cause reduced gene function should also increase seed protein content.

Introduction of the RNA GM-CCT CR4 is expected to knock out, silence or suppress expression of the glyma.10g134400 sequence on chromosome 10. Plants which have knocked out, silenced, or suppressed expression of the glyma.10g134400 polypeptide and showing increased oil content in seeds were selected. In some plants protein content was reduced.

### Example 4. Optimization of CCT-Domain Protein Expression to Minimize Pleiotropic Effect on Agronomic Traits

The expression patterns of glyma.20g085100 gene and its paralogue glyma.10g134400 were measured in developing soybean tissues and suspension cultures. Glyma.20g085100 was found to be expressed weakly in developing seeds, flowers, and leaves (Table 6).

TABLE 6
Expression of Glyma.20g085100, its paralogue glyma.10g134400,
and two homologs glyma20g200400 and glyma.10g190300

| | Glyma.10g134400 | Glyma.20g085100 |
|---|---|---|
| | Ratio of RNA/ | Ratio of RNA/ |
| Tissue/Cell | Total RNA (PPM) | Total RNA (PPM) |
| soy_embryogenic_suspension_culture (cell culture) | 0 | 0 |
| soy_cotyledons (cotyledon) | 50.36 | 17.22 |
| soy_somatic_embryos_germination (embryo) | 6.05 | 2.25 |
| soy_somatic_embryos_dry_down (embryo) | 1.32 | 0 |
| soy_somatic_embryos_maturation_SHAM (embryo) | 0.37 | 0.57 |
| soy_somatic_embryos_maturation (embryo) | 0.76 | 1.26 |
| soy_flower (flower) | 58.78 | 32.47 |
| soy_flower_cluster (flower) | 15.52 | 7.64 |
| soy_leaf_flowering (leaf) | 1.91 | 54.12 |
| soy_leaf_first_trifolate (leaf) | 9.81 | 5.03 |
| soy_shoot_apical_meristem (meristem) | 0.22 | 2.07 |
| soy_leaflet_petiole (petiole) | 10.23 | 7.11 |
| soy_main_petiole (petiole) | 10.48 | 5.88 |
| soy_pods_1cm (pod) | 20.2 | 12.18 |
| soy_pods_2cm (pod) | 9.43 | 5.31 |
| soy_root_seedling (root) | 1.44 | 0.67 |
| soy_root_tips_seedling (root) | 0 | 0.62 |
| soy_seed_50_DAF (seed) | 40.17 | 9.24 |
| soy_seed_30_DAF (seed) | 31.58 | 7.01 |
| soy_seed_15_DAF (seed) | 4.52 | 1.37 |
| soy_seed_50DAF (seed) | 114.7 | 18.71 |
| soy_stem (stem) | 4.01 | 1.01 |

To maximize the high protein phenotype while minimizing pleiotropic effects, a polynucleotide encoding a modified version of glyma.20g085100 with the insertion removed (SEQ ID NO:4) and/or a polynucleotide encoding glyma.10g134400 (SEQ ID NO: 6) are transgenically expressed in the seed under a seed-specific promoter. The modified glyma.20g085100 (without insertion) or glyma.10g134400 are each operably connected to a seed specific promotor that weakly expresses, such as soybean Gm-ALB promoter (2S albumin promoter, Glyma13g36400, NCBI Accession # gb AAE71140.1) or Gm-GA20OX promoter (GA20 oxidase, glyma07g08950, Lu et al). A terminator, such as the native terminator or soybean MYE2 terminator (transcriptional factor MYB21-related, glyma.19g061600) is operably connected downstream from the coding sequences. Vectors, containing expression cassettes such as shown in Table 7, are transformed into elite soybean 93Y21 via Ochro-based transformation such as described in Example 2. Transformation can be carried out for both glyma.20g085100—insertion removed and glyma.10g134400 together, or each sequence separately. When targeted together, the glyma.20g085100—insertion removed and glyma.10g134400 cassettes can be on the same or different constructs.

TABLE 7
Constructs/expression cassettes for transgenic expression

| Promoter | Gene | Terminator |
|---|---|---|
| Gm-ALB promoter | Glyma.20g085100 - | Gm-MYB2 Term |
| | insertion removed | |
| Gm-ALB promoter | Glyma.20g085100 - | Glyma.20g085100 |
| | insertion removed | Term |
| Gm-GA20OX promoter | Glyma.20g085100 - | Gm-MYB2 Term |
| | insertion removed | |
| Gm-GA20OX promoter | Glyma.20g085100 - | Glyma.20g085100 |
| | insertion removed | Term |
| Gm-ALB promoter | Glyma.10g134400 | Gm-MYB2 Term |
| Gm-ALB promoter | Glyma.10g134400 | Glyma.10g134400 |
| | | Term |
| Gm-GA20OX promoter | Glyma.10g134400 | Gm-MYB2 Term |
| Gm-GA20OX promoter | Glyma.10g134400 | Glyma.10g134400 |
| | | Term |

Transgenic seed oil and protein content is determined by SS-NIR and FT-NIR spectroscopy as described previously (Roesler et al Plant Physiol. 2016 878-893). Briefly, T2 homozygous seeds and null segregates are measured on a Bruker Multi-Purpose Analyzer FT-NIR spectrometer fitted with a 54-mm-diameter rotating cup assembly. Sample sizes of approximately 100 seeds (20 g) are used for the analysis. The weight of each sample (to an accuracy of 0.01 g) is recorded prior to scanning. The reflected spectra are captured for each sample to a wave number resolution of 8 cm-1 (1.5 μm) in the wavelength range between 833 and 2,778 nm, with the instrument in macro-reflectance mode. The cup is rotated over the source and detector while 64 full spectral scans are collected. The rotation of the cup is stopped, and the soybeans are poured into a foil pan and then returned to the cup prior to scanning for a second time. About three full-scan cycles (with complete mixing of the sample between each scan) are used. Captured spectra are analyzed, and models are used to predict moisture content, oil content, protein content, and oleic acid content using the Bruker OPUS 7.0 software package. The reference chemistry methods used for the calibration of moisture, oil, and protein are based on AOCS official methods (Ac 2-41 [moisture], Ac 3-44(mod) [crude fat/oil], and Ba 4e-93 [crude protein]). The reference chemistry used for the oleic acid calibrations utilizes gas chromatographic analysis of fatty acid methyl esters of oil extracts derived from the soybean samples, after spectral capture.

Field trials are carried out to measure the impact of seed-specific expression on agronomic traits and yield. A nested field experimental design is adopted to evaluate seed trait performance, where positive and negative blocks are nested within each respective event and positive and negative isolines were randomly nested within each positive and negative block, respectively. Recorded traits included the content of oil, protein, and oleic acid. Least-squares means for positive and null within each event are calculated using a mixed-model analysis method via the residual maximum likelihood software package ASRemI (Gilmour et al., 2009). Event and positive and null trait classes are treated as fixed effects, and isolines were fitted as random effects. The spatial variation of first-order autoregressive (AR) correlation structure for rows and autoregressive correlation for columns (AR1×AR1) is incorporated in the analysis. Mean differences of trait versus null were determined based on Fisher's Isd approach at a significance level of P<0.05. It is expected that high-yielding, high protein and high-oil plants and seeds are obtained expressing one or both of (i) the glyma.20g085100 with the insertion removed polypeptide and (ii) the glyma.10g134400 polypeptide.

### Example 5: Increase Seed Protein Content by Editing Glyma.10q134400 Promotor or Glyma.20q085100 Promoter

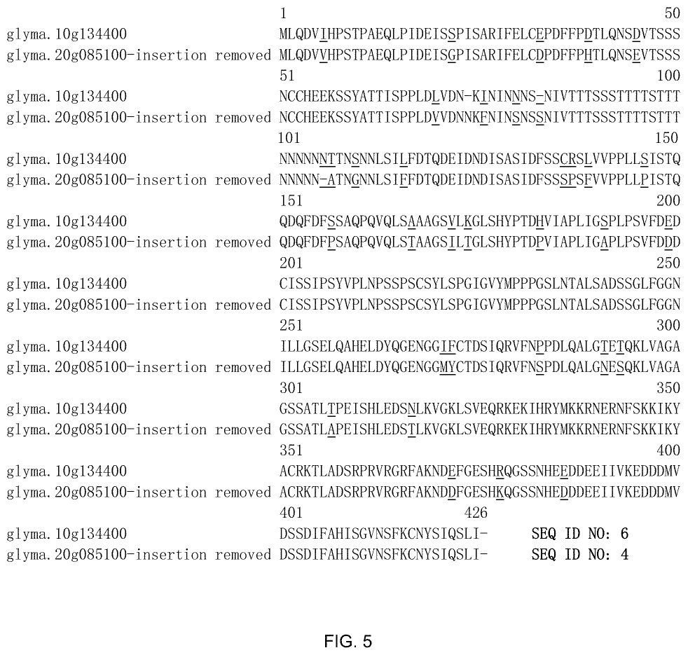

The 321 base pair insertion is removed from elite glyma.20g085100 gene according to Example 2. The resulting gene encodes a protein which shows 91.5% identify to its paralogue glyma.10g134400 (FIG. 5). To increase expression of glyma.10g134400 or glyma.20g085100 with the insertion removed, an EME (expression modulating element) is inserted or edited in the promoter region about 20 bp upstream of the TATA box of glyma.10g134400 or glyma.20g085100. The EME (expression modulating element) is a short fragment of DNA of about 16-50 bp which can enhance target gene expression when inserted in the target gene promoter (International Application No.: PCT/2018/044498; U.S. provisional application No. 62/558,619). Insertion of the 2×Zm-AS2 (SEQ ID NO: 23), an EME comprising a repeated sequence from maize into the soybean promoter region is expected to produce a 2- to 5-fold increase in gene expression. The modified promoter of glyma.20g085100 or glyma.10g134400 with 2×Zm-AS2 (SEQ ID NO: 23) can be cloned into a vector to drive ZsGreen1 fluorescence protein expression. The vector comprising the modified promotor sequence containing the EME sequence and the fluorescent marker is introduced into protoplasts by PEG mediated transfection. The 2×ZM-AS2 can be evaluated in protoplasts for expression modulation activity of glyma.20g085100 or glyma.10g134400 promoter using the green fluorescence protein as a reporter gene. Fluorescence level in protoplast can be measured as an indicator for promoter strength. The 2×Zm-AS2 EME constructs that show elevated expression are further tested in stable soybean transgenic plants or tested by editing the genomic sequence to include the EME in the transcription regulatory region near TATA box as described in Examples 2 and 3.

Deletion of repressor elements in the promoter region by CRISPR/Cas9 may also increase gene expression. Repressor elements in the promoter region can be identified using promoter or motif-based sequence analysis tools, such as The MEME Suite funded by the NIH and found online at meme-suite.org (University of Queensland, Australia, University of Washington, US and UC San Diego, US) or The Plant Promoter Analysis Navigator “plantPAN2.0” found online at plantpan2.itps.ncku.edu.tw/index.html (Institute of Tropical Plant Sciences, National Cheng Kung University, Taiwan). The repressor elements are deleted or suppressed using methods disclosed herein.

### Example 6. Identify CCT-Main Gene Mutants from Mutagenized Populations

Soybean mutagenized populations can be generated by gamma-ray irradiation, fast neutron irradiation, or chemical treatment with EMS (ethyl methanesulfonate) or ENU (N-ethyl-N-nitrosourea). Treatment of soybean seeds with 60 mM EMS can induce 5000-10000 mutations in a M2 plant. Each M2 plant can be sequenced by whole genome sequencing. Compared to wild type reference genome, all mutations in a M2 plant can be detected and mapped to genome. By sequencing about 2000-5000 M2 lines, it is possible to identify a mutation in a gene of interest in the soybean genome. A M2 line containing a mutation in glyma.20g850100 or glyma.10g134400 is identified, and is backcrossed to wild type soybean to clean up other mutations unrelated to CCT-domain gene. The mutants with high seed protein content can be crossed to other high protein mutants to generate double mutants which will increase seed protein content more than the increase from either single mutant.

1. A method for increasing protein content in the seed of a soybean plant, the method comprising introducing a modification into a CCT-domain gene in a soybean plant, wherein the modification is selected from:
a. a modification which comprises a deletion of nucleotides on chromosome 20 in a genomic sequence encoding a polypeptide comprising an amino acid sequence that is at least 95% identical to SEQ ID NO: 2, the deletion resulting in a modified genomic sequence on chromosome 20 that encodes a polypeptide comprising an amino acid sequence that is at least 95% identical to SEQ ID NO: 4;
b. a modification of a transcription regulatory sequence of a nucleotide sequence on chromosome 10 encoding a polypeptide comprising an amino acid sequence that is at least 95% identical to SEQ ID NO: 6, the modification resulting in an increase in expression of the polypeptide;
c. a modification comprising a first modification of part (a) and a second modification of a transcription regulatory sequence of a genomic sequence encoding a polypeptide comprising an amino acid sequence that is at least 95% identical SEQ ID NO: 4, the second modification resulting in an increase in expression of the polypeptide comprising an amino acid sequence that is at least 95% identical SEQ ID NO: 4;
d. a modification of one or more nucleotides on chromosome 20 in (i) a polynucleotide encoding a polypeptide comprising an amino acid sequence that is at least 95% identical to SEQ ID NO: 2 or (ii) a transcription regulatory sequence of the polynucleotide, the modification resulting in suppression of expression of the polypeptide; or
e. a modification of one or more nucleotides on chromosome 10 in (i) a polynucleotide encoding a polypeptide comprising an amino acid sequence that is at least 95% identical to SEQ ID NO: 6 or (ii) a transcription regulatory sequence of the polynucleotide, the modification resulting in suppression of expression of the polypeptide;
and growing the plant to produce a seed, wherein the protein content is increased in the seed, compared to a control seed of a control plant not comprising the modification.

2. The method of claim 1, the method further comprising crossing a plant comprising the modified CCT-domain polypeptide grown from the seed with a second different plant and harvesting the progeny seed.

3. The method of claim 1, wherein the modification comprises (i) a and b or (ii) b and c.

4. The method of claim 1, wherein the modification comprises the deletion of part (a), and wherein the deletion comprises a deletion of at least 312 and less than 330 nucleotides from position 6003 to 6358 of SEQ ID NO: 9.

5. (canceled)

6. The method of claim 1, wherein the modification comprises the modification of part (b) or part (c), and wherein the modification comprises an insertion of a promotor-enhancer element, an alteration of a repressor element, or a rearrangement of regulatory elements.

7. (canceled)

8. The method of claim 1, wherein modification comprises the modification of part (d) or part (e) and wherein the modification comprises (i) an alteration of the polynucleotide resulting in a frame-shift of the polypeptide coding sequence, or (ii) a disruption of a promoter-enhancing element, an insertion of a repressor element or a rearrangement of regulatory elements.

9. (canceled)

10. The method of claim 1, wherein the deletion or modification is introduced through targeted DNA breaks.

11. A plant having increased protein content, the plant comprising a modified CCT-domain genomic sequence, the modification selected from:
a. a modification which comprises a deletion of nucleotides on chromosome 20 in a genomic sequence encoding a polypeptide comprising an amino acid sequence that is at least 95% identical to SEQ ID NO: 2, the deletion resulting in a modified genomic sequence on chromosome 20 that encodes a polypeptide comprising an amino acid sequence that is at least 95% identical to SEQ ID NO: 4, wherein the plant produces seeds having an increased protein content relative to a control seed not comprising the deletion and a yield that is at least 80% of soybean variety 93B83 when grown under the same environmental conditions;
b. a modification of a transcription regulatory sequence of a nucleotide sequence on chromosome 10 encoding a polypeptide comprising an amino acid sequence that is at least 95% identical to SEQ ID NO: 6, the modification resulting in an increase in expression of the polypeptide, wherein the plant produces seeds having increased protein content relative to a control seed not comprising the modification;
c. a first modification of step (a) and a second modification of a transcription regulatory sequence of the genomic sequence encoding a polypeptide comprising an amino acid sequence that is at least 95% identical SEQ ID NO: 4, the second modification resulting in an increase in expression of the polypeptide comprising an amino acid sequence that is at least 95% identical SEQ ID NO: 4, wherein the plant produces seeds having increased protein content relative to a control seed not comprising the modifications;
d. a modification of one or more nucleotides on chromosome 20 in (i) a polynucleotide encoding a polypeptide comprising an amino acid sequence that is at least 95% identical to SEQ ID NO: 2 or (ii) a transcription regulatory sequence of the polynucleotide, the modification resulting in suppression of expression of the polypeptide, wherein the plant produces seeds having increased protein relative to a control seed not comprising the modification; or
e. a modification of one or more nucleotides on chromosome 10 in (i) a polynucleotide encoding a polypeptide comprising an amino acid sequence that is at least 95% identical to SEQ ID NO: 6 or (ii) a transcription regulatory sequence of the polynucleotide, the modification resulting in suppression of expression of the polypeptide, wherein the plant produces seeds having increased oil relative to a control seed not comprising the modification.

12. The plant of claim 11, wherein the modification comprises the deletion of part (a), and wherein the plant produces seeds having a yield that is at least 95% of soybean variety 93B83 when grown under the same environmental conditions.

13. The plant of claim 11, wherein the modification comprises the deletion of part (a), and wherein the deletion comprises a deletion of at least 312 and less than 330 nucleotides from position 6003 to 6358 of SEQ ID NO: 9.

14. (canceled)

15. The plant of claim 11, wherein the plant produces seeds having increased protein content relative to a control seed not comprising the modification.

16. The plant of claim 11, wherein the modification comprises an insertion of a promotor-enhancer element, an alteration of a repressor element, or a rearrangement of regulatory elements.

17. The plant of claim 11, wherein the plant comprises the first and second modifications of part (c), and wherein the second modification comprises an insertion of a promotor-enhancer element, an alteration of a repressor element, or a rearrangement of regulatory elements.

18. The plant of claim 11, wherein the plant comprises the modification of part (a) and wherein the deletion comprises a deletion of at least 312 and less than 330 nucleotides from position 6003 to 6358 of SEQ ID NO: 9.

19. (canceled)

20. (canceled)

21. (canceled)

22. (canceled)

23. A seed produced by the plant of claim 11, wherein the seed comprises the modification and has increased protein content relative to a control seed not comprising the modification.

24. (canceled)

25. A method of plant breeding, the method comprising crossing the plant of claim 11 with a second soybean plant to produce progeny seed.

26. A progeny seed produced by the method of claim 25, wherein the progeny seed comprises the modification and has increased protein content relative to a control progeny seed not comprising the modification.

27. A recombinant DNA construct comprising a heterologous promoter sequence operably connected to a polynucleotide encoding a polypeptide comprising an amino acid sequence that is at least 90% identical to SEQ ID NO: 4.

28. A soybean plant producing a seed comprising increased protein content, the plant comprising the recombinant construct of claim 27, wherein the polypeptide is expressed in the seed and the seed has increased protein content compared to a control seed not expressing the polypeptide.

29. A seed produced by the plant of claim 28, wherein the seed comprises the recombinant construct and has increased protein content compared to a control seed not expressing the polypeptide.

30. (canceled)

31. (canceled)

32. (canceled)

33. (canceled)

34. (canceled)

35. (canceled)

36. (canceled)

37. (canceled)


SEQUENCE LISTING (25 sequences)

SEQ ID NO: 1 - DNA, 1332 bp, Glycine max
atgttgcaggatgttgtccacccctcaacaccggctgagcaactccccattgatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaagtatgcttgccgcaaaattagagagagggttggagtagcgcctattgtagagaagatggtggaaaatagacttaggtggtttgggcatgtagagagaagaccggtagactctgtagtgaggagagtagaccagatggagagaagacaaacaattcgaggcagaggaagacccaaaaagactataagagaggttataaaaaaggatctcgaaattaatggtttggatagaagtatggtacttgatagaacattatggcggaagttgatccatgtagccgaccccacctag

SEQ ID NO: 2 - protein, 443 aa, Glycine max
MLQDVVHPSTPAEQLPIDEISGPISARIFELCDPDFFPHTLQNSEVTSSSNCCHEEKSSYATTISPPLDVVDNNKFNINSNSSNIVTTTSSSTTTTSTTTNNNNNATNGNNLSIFFDTQDEIDNDISASIDFSSSPSFVVPPLLPISTQQDQFDFPSAQPQVQLSTAAGSILTGLSHYPTDPVIAPLIGAPLPSVFDDDCISSIPSYVPLNPSSPSCSYLSPGIGVYMPPPGSLNTALSADSSGLFGGNILLGSELQAHELDYQGENGGMYCTDSIQRVFNSPDLQALGNESQKLVAGAGSSATLAPEISHLEDSTLKVGKLSVEQRKEKIHRYMKKRNERNFSKKIKYACRKIRERVGVAPIVEKMVENRLRWFGHVERRPVDSVVRRVDQMERRQTIRGRGRPKKTIREVIKKDLEINGLDRSMVLDRTLWRKLIHVADPT

SEQ ID NO: 3 - DNA, 1275 bp, Artificial Sequence
  Synthetic
atgttgcaggatgttgtccacccctcaacaccggctgagcaactccccattgatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaagtatgcttgccgcaaaactttggcggatagccggccccgggttagaggaaggtttgcaaagaatgatgactttggagagagccataaacaaggaagtagcaatcatgaagatgatgatgaagagataattgtgaaagaagatgatgatatggttgattcctcagatatctttgcacatatcagcggagtgaactctttcaaatgcaactattccatccagtccttgatttga

SEQ ID NO: 4 - protein, 424 aa, Artificial Sequence
  Synthetic
MLQDVVHPSTPAEQLPIDEISGPISARIFELCDPDFFPHTLQNSEVTSSSNCCHEEKSSYATTISPPLDVVDNNKFNINSNSSNIVTTTSSSTTTTSTTTNNNNNATNGNNLSIFFDTQDEIDNDISASIDFSSSPSFVVPPLLPISTQQDQFDFPSAQPQVQLSTAAGSILTGLSHYPTDPVIAPLIGAPLPSVFDDDCISSIPSYVPLNPSSPSCSYLSPGIGVYMPPPGSLNTALSADSSGLFGGNILLGSELQAHELDYQGENGGMYCTDSIQRVFNSPDLQALGNESQKLVAGAGSSATLAPEISHLEDSTLKVGKLSVEQRKEKIHRYMKKRNERNFSKKIKYACRKTLADSRPRVRGRFAKNDDFGESHKQGSSNHEDDDEEIIVKEDDDMVDSSDIFAHISGVNSFKCNYSIQSLI

SEQ ID NO: 5 - DNA, 1272 bp, Glycine max
atgttgcaggatgttatccacccctcaacaccggctgagcaactccccattgatgagatttcaagcccgattagtgctcgaattttcgaactttgcgagcctgatttcttcccagacacactgcaaaattcagatgttacttccagctcaaattgttgccatgaagagaagtcctcatatgccacaaccatatctccacctttagatttagtagacaacaagatcaatatcaataacaatagcaacatagtcactactacctcatctagcactaccacaaccagcaccacaaccaacaacaacaacaacaacacaacgaacagcaataacctgtccatcctctttgacactcaagatgaaattgacaatgacatctcagcctccatagacttctcatcatgtcgatctttagttgttccaccacttctctcaatctcaactcagcaggatcagtttgatttctcttcagctcagccacaggtgcaactatcagcagcagcaggttcagttttgaagggcctctctcactaccctacagatcatgtgattgcaccccttattggatctccgttaccatctgtttttgatgaagattgcatatcttccatcccttcttatgtgcctctcaacccatcatcaccctcttgctcttatctcagtcctggcataggagtgtacatgcctcctcctggttcccttaacactgccttatctgctgacagttctggattgtttggtgggaacattctactggggtctgaactgcaggcacatgaattggactatcagggagaaaatggtggaatattttgcacagattcaattcagagggtgtttaaccccccagatcttcaggcacttggtactgagactcagaaacttgtagctggggctggaagttctgccactttgacaccagaaatctcacacttggaggactctaacttgaaagttggaaaactctctgttgagcagaggaaggaaaagattcatagatacatgaagaagagaaatgaaagaaatttcagcaagaaaatcaagtatgcttgccgcaaaactttagcagatagccggccccgggttagaggaaggtttgcaaagaatgatgagtttggagagagccatagacaaggaagtagcaatcatgaagaagatgatgaagaaataattgtgaaagaagatgatgatatggttgattcctcagatatctttgcacatatcagtggagtgaactctttcaaatgcaactattccatccagtccttgatttga

SEQ ID NO: 6 - protein, 423 aa, Glycine max
MLQDVIHPSTPAEQLPIDEISSPISARIFELCEPDFFPDTLQNSDVTSSSNCCHEEKSSYATTISPPLDLVDNKININNNSNIVTTTSSSTTTTSTTTNNNNNNTTNSNNLSILFDTQDEIDNDISASIDFSSCRSLVVPPLLSISTQQDQFDFSSAQPQVQLSAAAGSVLKGLSHYPTDHVIAPLIGSPLPSVFDEDCISSIPSYVPLNPSSPSCSYLSPGIGVYMPPPGSLNTALSADSSGLFGGNILLGSELQAHELDYQGENGGIFCTDSIQRVFNPPDLQALGTETQKLVAGAGSSATLTPEISHLEDSNLKVGKLSVEQRKEKIHRYMKKRNERNFSKKIKYACRKTLADSRPRVRGRFAKNDEFGESHRQGSSNHEEDDEEIIVKEDDDMVDSSDIFAHISGVNSFKCNYSIQSLI

SEQ ID NO: 7 - DNA, 1254 bp, Glycine soja
atgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaagtatgcttgccgcaaaactttggcggatagccggccccgggttagaggaaggtttgcaaagaatgatgactttggagagagccataaacaaggaagtagcaatcatgaagatgatgatgaagagataattgtgaaagaagatgatgatatggttgattcctcagatatctttgcacatatcagcggagtgaactctttcaaatgcaactattccatccagtccttgatttga

SEQ ID NO: 8 - protein, 417 aa, Glycine soja
MLICHHSLQDEISGPISARIFELCDPDFFPHTLQNSEVTSSSNCCHEEKSSYATTISPPLDVVDNNKFNINSNSSNIVTTTSSSTTTTSTTTNNNNNNATNGNNLSIFFDTQDEIDNDISASIDFSSSPSFVVPPLLPISTQQDQFDFPSAQPQVQLSTAAGSILTGLSHYPTDPVIAPLIGAPLPSVFDDDCISSIPSYVPLNPSSPSCSYLSPGIGVYMPPPGSLNTALSADSSGLFGGNILLGSELQAHELDYQGENGGMYCTDSIQRVFNSPDLQALGNESQKLVAGAGSSATLAPEISHLEDSTLKVGKLSVEQRKEKIHRYMKKRNERNFSKKIKYACRKTLADSRPRVRGRFAKNDDFGESHKQGSSNHEDDDEEIIVKEDDDMVDSSDIFAHISGVNSFKCNYSIQSLI

SEQ ID NO: 9 - DNA, 8035 bp, Glycine max
tgtgtaaccaaagtctgttagtttaaaaaaaaattaattgtacccaaaaaataatataagtataaattggtacccaaaaaataatataagtatatatacatatataggtcggtctatcaggcttataaggctttctaataagcctaagtctggtttatttaatttaataggcttttaaaaaagtttgaacctaacattttaattaaataagtcagtccaggtcagattttatgtaggccaagtcgtagacctctgtaggccggcctagcttattctcacccctaattaagacaagctgtaggtcggcccctgttcatgttgttcatcaattattgtttaaccgagttaaaggagatggtcaattttacagtagtaaatatgacaagaaatacacgaaatttgtgtttaagtattgaaggtcttccatttttttgaatccaccatcctaaaactaatagtgtctaattttcccgtaacaatttcttttgtataacattcaaataaatgtggttgacgatatatatttagcttgcaaatttcctaaaaattattgctccaaacttagtatgcacttaggattggattgtgtcattattgtacttggcaagggtacatgtgttaattatatgcacttaagcgtgtcattgtacttggctacttaaaattgttttttgttcctcggatgatgttgatgagttataaaaaagaaatatatgccttgaaggaaagaaagtttatttttacggtttatgttaaaatacaatactttcaactatgtagattgtggagcaaacaagctttcagtttctctatcgtgttccgatgacacgttaataatgaacttttcctataaacatcaattagaagatgttgattttgttggtcaattggtgttattgttgtgactactttatattttcgttagattccaagcattatttccgtctatagggtttgctcttaactgattgttttgagttattaattattcaagttgatttgattggaatagtaaatctggatctattatgtaatatattagggacgaaatcttgcacattgagaatgatgtagtctcctcaaaattttgataaccattgttttcgagtgatagatgttgtctatgtcaaggttgtcgatgaaattagcaatgatggacagactttgtcgaacaagttttgtaatgccatcacagaaaagcatctagaagtactgaaaaacattttttttaacatatacttttacaaaatcaagagaatgaataattaaatatgaaataagaattacaaaaattttatatttttttaataaatttcaaatcaataataagaaagaatgggataaaattaaagaatatgttggaagatgtgttattagtatttctgtgcataagaatgcttccaaaattaaactctttccccaaaattgtatgagtaaaatttcaaatgtctcgttttaaatataacataagctaaattaaacccagcttagtaaaaaaaatgcatttccgtaaatttcgatgtccaaacaattgtaatgacttaaactcagaacaaaacctatttttataccatgtaaattcttacacattataatcgtatgtttatccatccatgcaccccaacaaatgaagaaaaggaataaaaaaaaaggtgaatttgccaaaaacaaattaatgggatgctaacattaacataaggcggcatagggcatcatttaattgtgaataaaaaggcttggtcaacccaatttcttctcctagttactagttattaaccatgtcatttggcatatatataataatccatatccatataaaaatacatgtcccaaaaattgaattccactacaccccaccaaagtggtgaattaatggccaaacgccactctcgcgttgacacccgcgagtgccaggcttgacgactctacgtacaacctcatcaattcctctatatatgcacataactaactttctctgtttttgacaccctcatacaccccatcatcttagctaacacaacacagcatacacaacttctctctctctactactttctttttatgtcaccttcttgatgtctttaatttgttttcttttttagaaaaaaattaatcctttttaaggctttaatttcctaagcatgcaatattattgtttttagtcaccttcaagttgaggaacatatacacgtttgtgaccacaccaaattccacttctttctaagtgtgtgtacatagattttgttttatatatatttgaggttcacccttcattgcctccttaattctgtgcaaaagaggattcatcaccaccatgttgcaggatgttgtccacccctcaacaccggctgagcaactccccattgtaatcacctcaagcttgttacatttcatatctcgatcatgtatatttagtgttaatgcatgtattggaactaagctatatatgtatgtgtgtatgtatctttgtgtcatgcaatatttatgaccacagctaaatggtttccttctgggttttgtgatcgtgtgtaacttgacaagtttgtttggatcaagggcttgtttttttctttcaaaataatgatttcacactccaagagtgtgtaaaccttgggagggaagagaaagacaggaaagagaccccagaaaagaacaaaagaagtctaaaattgatgtacggattaatagcgtgtttgaaaacccattgagaagtaagaaaatctcatctgagatatgaaagttatgactattaacttcctaataaactcaagaaatcacatctgagatgtgaaaccaaacatgctcttacgaattgtagaaattttgatggtttttctctggggtactctttcttatattttgaggccaattattactaatttctctaggaattattcgatcaattgctccattcctgcattttaattctttccatgcatgaatcacgaatatggttttgttaatgtttgttggcatgttaattaatttcatcctaattagttctctgagaaacctgaacaatatttcccttatttggttatattcctggagctaagtggaagctgctagtgctattagccatcttagaaaacccacaagccatgctcactatttgtgggacggaacgttttctttcctctatttatatggttggagagcataacgtaattttattgaccgaacaataggaaggaatcaaagactccgtgaccgtgctgtcaactcttttgatatttattatcatccaatgggccctaccaccaaaatgtttaatgtcattttccaaggggcaaagttgtaaaaatgatgtaacatttccgtgatagtaatttagacatagctttcggttcttgtagaatatatatatctctttgggggcttgttgtgtaagctttgctaatttttgatattctttattcagttagatagggagaagaggtcttagtgacaaacaattcatatggatagcatatatagcattatggggttctcttcaagtggctttcaattcccatttaagttcaaattttccacttaaaatgaacaaaaataacatagcatgaggttccttgtttgtttttgcatacttgtatttcatactaacattggctaccttggctcctacctgtcacatgaaggcatagatgcacatcactttcaccaataaataacccaacaatgcaaaccctttaggaaaatttctcttcagtctattaccaaatatggacaaaactgactagactatgcaaccaaccacagtcatatgtagaattcttcgttggggcctttgcacatcgatttatattgttttcctcttgataaagaaggaagggggtgttgtggcagatactattatatcatttggcatgtctttcacttttgaatgcccactacatctacaagccaaagcttcaattgattagagaatattagcttttgaatttcttttctgttctaagtgatcagatcagactcatgtctatcaacaaaaagaggaattggattcagatttcaaacccatcataatgaagaaaaaaataaaaaaaataaactatagagaatcacgtttcctagattgtttataaagaagaggctttgttaagactattctcaggctgtgttttagtagtttatggtaaacatgttgttcacaatgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggtatgtgcaatttcgcaagccaattagagtttaatagacattcattgtctggtataaaagtttttacattatcaatcaatcagataccattgttgatataaattttaaaataattgttataataattaataatttaatgtacttgataatttgtgatttgataataatataaaaaaaatttacactgcattattatttatttttcctgtcgactgtcaataaactaaatgaaaattttcagttccaatgttcaatgtgttcagaataaggaaaaagaagtttaataatgctgcaaaggttactataccttgcagcagtgaagtttttattttaaaatagaagaggctttatcagaggtggacttttgggggaaagctcagggtccacaaatctctaaactataaactcataggtgccccatgaccatcaaatagtaggtagcacaagatatgagtccatttataaagtcacatgcattaaaaaatactataaatttggcctagcaagaaggaagaaccactttcatccaaaagaaaaatagaaaaaaggataataaactgtagcatcattagatagaaagacccacttcaagggtggcagtgttatatctctttctacagtctataaagttaatgtgcagtttttattgaataagtaagaaattgatctttaattataatttctctctcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaaggtactacatctgaacaccaacattaacaaacaaatttcaaatcttatactgttttacatgatttccaatctactgcatcaaccaagccttatgcatattttcaaaattcaactaatgatgcaattttttttatataaaaaaaatgcagtatgcttgccgcaaaattagagagagggttggagtagcgcctattgtagagaagatggtggaaaatagacttaggtggtttgggcatgtagagagaagaccggtagactctgtagtgaggagagtagaccagatggagagaagacaaacaattcgaggcagaggaagacccaaaaagactataagagaggttataaaaaaggatctcgaaattaatggtttggatagaagtatggtacttgatagaacattatggcggaagttgatccatgtagccgaccccacctagtgggataaggcgttgttgttgttgttgttgttgtatgcttgccgcaaaactttggcggatagccggccccgggttagaggaaggtttgcaaagaatgatgactttggagagagccataaacaaggaagtagcaatcatgaagatgatgatgaagaggtaagattcccttaatcggatactgttgttcaacttgccttagtctaaaaattaaaatacaaaaaaattcccgatcacttttaccttttcaattatttgatggcataattccttgatgttatattccttccattttttgtacttgcagataattgtgaaagaagatgatgatatggttgattcctcagatatctttgcacatatcagcggagtgaactctttcaaatgcaactattccatccagtccttgatttgaattaaattattagtttgactagtgaaagcttatttatataattagcttctgtagattaattttggtaggacacttttcccatcccggttctctaaaatccgggtttagtggtttgagtaaactgaataaatggggtcaaaataaatataccaataagttaagtgagttagaaacgtacagaaattggaaactgtatacatttttgcagatatatattatctttttcattaagttgtaccagaacatggagttgtgttaaccaagaaaatttccagttacccccatccaagactgatgtaaccaattgatgtagcttcttttataaatatttaggaacttgcttttaaggttttttttttttttgatgatgggttgcttttaagtaattttacatcctctaattatttttttcttaaatatgggattaaattgattgttacttgttgaagctaaaaaaggtttataatgttatggactaaattgatgttgtattgatttattggttcaactaaaataagaatataatggtaacacaataataatatcatttactcgtaaattattcttggtataatttttaaaatgattattataaaaatcaacaaaattattatatatgatgagttataattagatgaggtatatattttacaccgtgaatgtttccttattttcttaaaaataaaatgatggtaaaccttaaatcctatagtagcgctaaactaggttaagcttgcaactcttattcgctaacctggtgacaacagaactcttttgtttggacatttgcctagtaaagattagaagaggtccacaatggatggaaaggtacagttatacttctatttcggtaacttttagaatatttggcaaaattctcactaaacttgtagaatactttattcgttaaatagtacagttatctttttttttcaatgcaaaataatttaattgtcgaacataactttcaagagataaatgatttctacttacacggggaggataattgaatgtgggatttttttttattttacttctttagttctttatgggaaagaacttttaattaattcagaattcgatcataatttcgttaaagatcaaatatcaaatgattcaatcttaattttaatacattaattatttattataacgtgatttgatctcatattttttctatggtcaataaaatattggctaaatgatacgtgtagtcttttatgttattgtttagatttaatttaattatttatcttttaaatttagtttcatttaatcattctgcccgtttaaaattaatgttgttaataattaacatatcga

SEQ ID NO: 10 - DNA, 7871 bp, Glycine max
aaagaagtcagtggaagatttttcaaattgaaataaaaaaaatacgaatcttctactccttaaagaatttgggaaaaggggtagagtgattatacatgcatttgaaataaaaaaacacacacgccaaagagcccgttaatattggccacagggtggtgttgacgagttataactatgtgccttgaaggaaagaagtttttttttttaaaaaaaaaaaaaaaaaagccaaatggtattataaacaacaaacaaagcacaagtagtgcctaatacaagaaaagggatcaagaatgttacctgacccacatacgaaaatcatttagagcccacatacatcccatactatgatcaaaatagcgcatcatggaataaaaatgcaacagaacactatatgtaaaccatgctaaacttaaagctgctcccattgatgaaaataattgctctccacagctccataaaaatcttgtgtcacctacttaggccggaacacttcgagaaaaaccaaataccacgagcaagggctctacaaacattgccacggagaattacaccaacatgaaactggataaatataaccaggattccctttcagccattgccaagtgatcaggagtattacttgctctagcataccccatacatctatcctttttccatatgtttaaggtttgtgttaaaatacaatatttcaacttatatactcgtgtagcaaacaagctttcaatttctcaatcgtgttgagatgactcattgataatgaactttttctataaacgtaaatcataagatgctggtgttgttggttaattggtgttattgttgaccactttatattttcattattagattctaaacattttctctgtctatagggtttgctcttaactgattgttttgagttattaattattcaagttgatttgattggaattgtacatttggatctattatgcaatatatttagatttttttaaattatagagtttaatatttgttattcttcaatttagggtgaaatcttgcacaatgagaatgatgtagtctcctcaaaattttgaacataattgttttcaagtgataaatatgttgtctatgtgaagattgtcgatgtacttaatgatggacatattttatccaacaagttttgtaatgtcatcaatgaaaagcatgaatagtatttatatggagagaaatgcttataacatactctttttaacacatttttttttattggttaaaagttattaaaaattataaaaaaaaattaaatatgaaatggggtccacaaaattttttgtttttgataaatttcagttaataaaaaagaatgtccaaaaaaatgtactacaaaaattgtgttactaatatttattttcgtaagaatgcttctaaaaattaacattttttcaaaagttgtattaataaaattttaaatttctcaatttaaatataaaataagctaaattaaacccaactaattagtgaagaaagaaaacatttccataaattttcgatgtcgaaacaattgtaatgacttaaactcaaaacaaaacctatttttaaaccatgcaatttcttacacattataatcctacatgtgtatccatccatgcaccgcaacaaatgaagaaaaggaataaaaaaaaaggtgaatttgccaaaaacaaattaatggaatgctaacattaacataatgcgacataggacagcatatttaattgtgaattaaaaggcttggtcaacccatattcttctcatagttactagttactagttagccatgtcatttaattggcatatatataataatccatatataaatatacatgtcccaaaaattgaattccactacaccccaccaaagtagtgaattaatggccaaacgccactctcgcgttgacgcccgcgagtgccaggcttgacggcctacgtacaacctcatcaattcctctatatatgcacataaccaactttctctgtttttgacaccctcaaacaccccatcatcttatagctaacacaacacaacttctctctttttctctctcttcctaccactttaatttcgtttcatgtcaccttcttgttttcttttttagaaaaattaatccctttaaggcttaattttctaattaagcatgcaatattgtttttaattagtcaccttcaagtcgaggaacatacatatacacatatgtttgtgatcaccacaccaaattccacttctttctaagtgtgtgtatgtgtgtacatagattttgttttatatatatttgaggtttcaccttcattattgcctccttaattctgtgcaaaagaggattcaccaccaccatgttgcaggatgttatccacccctcaacaccggctgagcaactccccattgtaatcacctcaagcttgttacatttcatgtctcaatcatgtatgtttagtattaatgcatgtggtggaggaactaagatatatatatatatatatatatatatctttgtgtcatgcaatattttttaccttagctaaatggtttccttctgggttttgtgattgggtgtaacttcacaagtttgtttagatcaaggctttttttttctttcaaaataatgatttcacactcaaagagtgtataaaccttgggagggaagacaaagataggaaagagaccccagaaaaagaaaaaaaaaaaaaaagtctaagattgatgtaattaataatgtgtataaacctcgtaaggattaattgcaagcgtgtgtgaaaacccgttgagaagtaagaaatcacacctaagatgtgaaagttgtcaattaacttcttaataaacgtgaaaaaccacgtctgaaagtgaaatcaaacacagtctttctcatgaggcctgaaaattttgatggttttttctcctgggtgctctttcttatattttgtgaccaattattacaaatttctctaggaattaatcaattaattgatccagtcctgcattttaattctttccatgtatggatgatgaatatggttttgttaatgtttgttggcatgttaattagtttcatcctaaattagtctctgagaaacctgaacaatatttcccttatttggttatattcctggagctaagtggaagctgctaatgctattattcatcatagaaaacccacaagccatgctcacaatttgtgggacggaacgttttctttcctctatttatatggttggagagcataacgtaattttattgaccgaacaataggaaggaattaaagactccgtgagagtgaggatcaactctttttttatattcattatcatccaacggccctaccaccaaaatgcttaatgtcattttccaagggacaaagttgtaaaaatgatgtaacatttccgtgatagtaatttagacatagctttcggttcgtgtggaatatatatctctttgggggcttcttgtgtaagctttgctaattttttttatattctttattcagttagatagggagaaggaagggtctttgtgacaaacaattcacatggatagcatatatagcattattgggttcccttcaagtggctttcaattcccattgaaggacaattttttccacttaaaatcaacaaaaataacatagcatgaggttccttatttgtttttgcatacttgtatttcataataacattggctaccttggctcctacctgtcacatgaaggcatagatgcacatcactttcaccaataaattacccaacaatgcaaacccttttggaaaactgctctcaagtctattaccaaatatggacaaaactgactagactatgcaaccaaccacagtcatatgtagaattctttgttggggcctttgcacattgatttatatcgttttcttgttgataaagaaggaaggggtggtgttgtggcagatactataatatcatttggcatgtctttcacttttgaatgcccactacatctacaagccaaagcttcaattgattagagaatattagcttttgaatttcttttctgttctaagtgatcagatcagactcatgtctatcaacaaaaataggaattggattcagatttcaaacccatcataatgaagaaaataaaataaataaaaataaactagagagaatcacgtttcctagtttgtttttataaagaagaggctttgttaagactatttctcagactatgttttagtagtttatatggtaaacatgttgctcacaatgcttatatgtcaccactcactgcaggatgagatttcaagcccgattagtgctcgaattttcgaactttgcgagcctgatttcttcccagacacactgcaaaattcagatgttacttccagctcaaattgttgccatgaagagaagtcctcatatgccacaaccatatctccacctttagatttagtagacaacaagatcaatatcaataacaatagcaacatagtcactactacctcatctagcactaccacaaccagcaccacaaccaacaacaacaacaacaacacaacgaacagcaataacctgtccatcctctttgacactcaagatgaaattgacaatgacatctcagcctccatagacttctcatcatgtcgatctttagttgttccaccacttctctcaatctcaactcagcaggatcagtttgatttctcttcagctcagccacaggtgcaactatcagcagcagcaggttcagttttgaagggcctctctcactaccctacagatcatgtgattgcaccccttattggatctccgttaccatctgtttttgatgaagattgcatatcttccatcccttcttatgtgcctctcaacccatcatcaccctcttgctcttatctcagtcctggcataggagtgtacatgcctcctcctggttcccttaacactgccttatctgctgacagttctggattgtttggtgggaacattctactggggtctgaactgcaggcacatgaattggactatcagggagaaaatggtggaatattttgcacagattcaattcagagggtgtttaaccccccagatcttcaggtatgtgcaatttttcaagctaattagcatttaataggcatgtattgttagtgtaaatttttttacatattgtcaatcaattaaaaattattattgataaaacttttaaaataattattattaaaattaacgaactatcatacataacaattgtgattcagtactaatgtaaaaatctttacatgtcaatgagtatattttatttattctttttgtcaactgtcaagggactaaatgagaattttcaattccaatgttccatgtgttcagaaataaggaaaaagaggtacaatggtcaaagaagtttattaatgctgcaaatgttactataccttgcagcagtgaagtgtttttttataaattagaagaggctttatcagaggtggacttttgggggaaagctcagggtccacaaatctctaaactataaactcataggtgccccatgaccatcaaatagtaggtagcaaaagatatgagtccctttataaagtcaaatgcattaaaaaatactaaaatttggcctagcaagtaggaataaccactttcagccaaaagaaaaacagaaaaaaaggatcacaaacagtagcatcattagatagaaagacccacgtcaagggtggctgtgttatatctctttctaaagtctctataaagttaatgtgcagtttttaatagtgtgtgggccaacatctttccactttgtgttgaataagaagtaagaaatttatctttgattataatgtctctctcaggcacttggtactgagactcagaaacttgtagctggggctggaagttctgccactttgacaccagaaatctcacacttggaggactctaacttgaaagttggaaaactctctgttgagcagaggaaggaaaagattcatagatacatgaagaagagaaatgaaagaaatttcagcaagaaaatcaaggtactacatctgaacaccaacattaacaaacaaatttgaaatcttatattatgttatacatgatttccaatctattgcatcaatcaagccttgtgcatattttcaaaattcaactaatgatccaatgttttttaaaaaaaaaatgcagtatgcttgccgcaaaactttagcagatagccggccccgggttagaggaaggtttgcaaagaatgatgagtttggagagagccatagacaaggaagtagcaatcatgaagaagatgatgaagaagtaagattcccttaattggatacttttgttcaacttgccttagtctaaagttaaaatacaaaaaaattccttatcacttttaccttttcaattatttgatggcataattccatgatgctatatcccttccattttttgtacttgcagataattgtgaaagaagatgatgatatggttgattcctcagatatctttgcacatatcagtggagtgaactctttcaaatgcaactattccatccagtccttgatttgaattaaaattaactattagtttgactagtgaaagcttatctatataatcagcttctgtagattaattttggcagggcccttttcccatcccggttctctacaaatccgggtttagtggcttgaggaaactgaataattgaggtccaaataattataccaataagtgaagtgagttaggaacgtacagaaattagaaactgtgtacatttttgcagatatatattatctttttcattaagttgtaatcgaacatggagttgcgttaactaaggaaaattccagttgccccctcccaagattgatgtagcttctttttataaatatttaggaacttgcttttaagtagctttacatgctctaattattctttctacttaaatatgattgataaatattgaagctgagaattgttataatgttacgaattaaattaatagataacgtttataatgttacggactaaattgatgttgcgttgatttattggtttagtttaaggtggcttgaatctgatttgggtactcatattatatattatatagttaaaataagaatttaatagtaatgtagtaataatttaatgttttatattatcgtttaattataaattatcgttaacttttaaattgattaccataaaattaataaatttatttataatatataaaagttgaacttctcaacattaattatttaaaagaatgaaaaagaaacattttgtatcactatattaattaaaataacactcatgaatagtctttgatatattttagtaaaatcaataacttctcatattaatattaatttaaattatttgtaatatttaattttacgtgttgaattaatatgaatgaaaaaatattaaataaaataaatgtaaaagtttacactaatttaatattattatatatgcataaatttatttatttttataattaattactttaaaatatatttttggaaaaataataaattaaattcatgactttaaagttataaaataatatagtataatataaaaatataaaaattatatatatatatatatatatatatatatataaattaaattattaacatttatatttaatgttaaatatttaaattagaactaattagataaatgtaaattaatgatagaagactaatagttaatataaatataaggatttttatattatttttattgtaaattttattttataaactatttttaaaaaaactataaatgtaataattaaattataattattattggcttcagttttatataaaaatagctatatgtaaaaatatatgttacaaaatttatggtatgataaagttaataatatttttcaatttta

SEQ ID NO: 11 - DNA, 7714 bp, Glycine max
tgtgtaaccaaagtctgttagtttaaaaaaaaattaattgtacccaaaaaataatataagtataaattggtacccaaaaaataatataagtatatatacatatataggtcggtctatcaggcttataaggctttctaataagcctaagtctggtttatttaatttaataggcttttaaaaaagtttgaacctaacattttaattaaataagtcagtccaggtcagattttatgtaggccaagtcgtagacctctgtaggccggcctagcttattctcacccctaattaagacaagctgtaggtcggcccctgttcatgttgttcatcaattattgtttaaccgagttaaaggagatggtcaattttacagtagtaaatatgacaagaaatacacgaaatttgtgtttaagtattgaaggtcttccatttttttgaatccaccatcctaaaactaatagtgtctaattttcccgtaacaatttcttttgtataacattcaaataaatgtggttgacgatatatatttagcttgcaaatttcctaaaaattattgctccaaacttagtatgcacttaggattggattgtgtcattattgtacttggcaagggtacatgtgttaattatatgcacttaagcgtgtcattgtacttggctacttaaaattgttttttgttcctcggatgatgttgatgagttataaaaaagaaatatatgccttgaaggaaagaaagtttatttttacggtttatgttaaaatacaatactttcaactatgtagattgtggagcaaacaagctttcagtttctctatcgtgttccgatgacacgttaataatgaacttttcctataaacatcaattagaagatgttgattttgttggtcaattggtgttattgttgtgactactttatattttcgttagattccaagcattatttccgtctatagggtttgctcttaactgattgttttgagttattaattattcaagttgatttgattggaatagtaaatctggatctattatgtaatatattagggacgaaatcttgcacattgagaatgatgtagtctcctcaaaattttgataaccattgttttcgagtgatagatgttgtctatgtcaaggttgtcgatgaaattagcaatgatggacagactttgtcgaacaagttttgtaatgccatcacagaaaagcatctagaagtactgaaaaacattttttttaacatatacttttacaaaatcaagagaatgaataattaaatatgaaataagaattacaaaaattttatatttttttaataaatttcaaatcaataataagaaagaatgggataaaattaaagaatatgttggaagatgtgttattagtatttctgtgcataagaatgcttccaaaattaaactctttccccaaaattgtatgagtaaaatttcaaatgtctcgttttaaatataacataagctaaattaaacccagcttagtaaaaaaaatgcatttccgtaaatttcgatgtccaaacaattgtaatgacttaaactcagaacaaaacctatttttataccatgtaaattcttacacattataatcgtatgtttatccatccatgcaccccaacaaatgaagaaaaggaataaaaaaaaaggtgaatttgccaaaaacaaattaatgggatgctaacattaacataaggcggcatagggcatcatttaattgtgaataaaaaggcttggtcaacccaatttcttctcctagttactagttattaaccatgtcatttggcatatatataataatccatatccatataaaaatacatgtcccaaaaattgaattccactacaccccaccaaagtggtgaattaatggccaaacgccactctcgcgttgacacccgcgagtgccaggcttgacgactctacgtacaacctcatcaattcctctatatatgcacataactaactttctctgtttttgacaccctcatacaccccatcatcttagctaacacaacacagcatacacaacttctctctctctactactttctttttatgtcaccttcttgatgtctttaatttgttttcttttttagaaaaaaattaatcctttttaaggctttaatttcctaagcatgcaatattattgtttttagtcaccttcaagttgaggaacatatacacgtttgtgaccacaccaaattccacttctttctaagtgtgtgtacatagattttgttttatatatatttgaggttcacccttcattgcctccttaattctgtgcaaaagaggattcatcaccaccatgttgcaggatgttgtccacccctcaacaccggctgagcaactccccattgtaatcacctcaagcttgttacatttcatatctcgatcatgtatatttagtgttaatgcatgtattggaactaagctatatatgtatgtgtgtatgtatctttgtgtcatgcaatatttatgaccacagctaaatggtttccttctgggttttgtgatcgtgtgtaacttgacaagtttgtttggatcaagggcttgtttttttctttcaaaataatgatttcacactccaagagtgtgtaaaccttgggagggaagagaaagacaggaaagagaccccagaaaagaacaaaagaagtctaaaattgatgtacggattaatagcgtgtttgaaaacccattgagaagtaagaaaatctcatctgagatatgaaagttatgactattaacttcctaataaactcaagaaatcacatctgagatgtgaaaccaaacatgctcttacgaattgtagaaattttgatggtttttctctggggtactctttcttatattttgaggccaattattactaatttctctaggaattattcgatcaattgctccattcctgcattttaattctttccatgcatgaatcacgaatatggttttgttaatgtttgttggcatgttaattaatttcatcctaattagttctctgagaaacctgaacaatatttcccttatttggttatattcctggagctaagtggaagctgctagtgctattagccatcttagaaaacccacaagccatgctcactatttgtgggacggaacgttttctttcctctatttatatggttggagagcataacgtaattttattgaccgaacaataggaaggaatcaaagactccgtgaccgtgctgtcaactcttttgatatttattatcatccaatgggccctaccaccaaaatgtttaatgtcattttccaaggggcaaagttgtaaaaatgatgtaacatttccgtgatagtaatttagacatagctttcggttcttgtagaatatatatatctctttgggggcttgttgtgtaagctttgctaatttttgatattctttattcagttagatagggagaagaggtcttagtgacaaacaattcatatggatagcatatatagcattatggggttctcttcaagtggctttcaattcccatttaagttcaaattttccacttaaaatgaacaaaaataacatagcatgaggttccttgtttgtttttgcatacttgtatttcatactaacattggctaccttggctcctacctgtcacatgaaggcatagatgcacatcactttcaccaataaataacccaacaatgcaaaccctttaggaaaatttctcttcagtctattaccaaatatggacaaaactgactagactatgcaaccaaccacagtcatatgtagaattcttcgttggggcctttgcacatcgatttatattgttttcctcttgataaagaaggaagggggtgttgtggcagatactattatatcatttggcatgtctttcacttttgaatgcccactacatctacaagccaaagcttcaattgattagagaatattagcttttgaatttcttttctgttctaagtgatcagatcagactcatgtctatcaacaaaaagaggaattggattcagatttcaaacccatcataatgaagaaaaaaataaaaaaaataaactatagagaatcacgtttcctagattgtttataaagaagaggctttgttaagactattctcaggctgtgttttagtagtttatggtaaacatgttgttcacaatgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggtatgtgcaatttcgcaagccaattagagtttaatagacattcattgtctggtataaaagtttttacattatcaatcaatcagataccattgttgatataaattttaaaataattgttataataattaataatttaatgtacttgataatttgtgatttgataataatataaaaaaaatttacactgcattattatttatttttcctgtcgactgtcaataaactaaatgaaaattttcagttccaatgttcaatgtgttcagaataaggaaaaagaagtttaataatgctgcaaaggttactataccttgcagcagtgaagtttttattttaaaatagaagaggctttatcagaggtggacttttgggggaaagctcagggtccacaaatctctaaactataaactcataggtgccccatgaccatcaaatagtaggtagcacaagatatgagtccatttataaagtcacatgcattaaaaaatactataaatttggcctagcaagaaggaagaaccactttcatccaaaagaaaaatagaaaaaaggataataaactgtagcatcattagatagaaagacccacttcaagggtggcagtgttatatctctttctacagtctataaagttaatgtgcagtttttattgaataagtaagaaattgatctttaattataatttctctctcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaaggtactacatctgaacaccaacattaacaaacaaatttcaaatcttatactgttttacatgatttccaatctactgcatcaaccaagccttatgcatattttcaaaattcaactaatgatgcaattttttttatataaaaaaaatgcagtatgcttgccgcaaaactttggcggatagccggccccgggttagaggaaggtttgcaaagaatgatgactttggagagagccataaacaaggaagtagcaatcatgaagatgatgatgaagaggtaagattcccttaatcggatactgttgttcaacttgccttagtctaaaaattaaaatacaaaaaaattcccgatcacttttaccttttcaattatttgatggcataattccttgatgttatattccttccattttttgtacttgcagataattgtgaaagaagatgatgatatggttgattcctcagatatctttgcacatatcagcggagtgaactctttcaaatgcaactattccatccagtccttgatttgaattaaattattagtttgactagtgaaagcttatttatataattagcttctgtagattaattttggtaggacacttttcccatcccggttctctaaaatccgggtttagtggtttgagtaaactgaataaatggggtcaaaataaatataccaataagttaagtgagttagaaacgtacagaaattggaaactgtatacatttttgcagatatatattatctttttcattaagttgtaccagaacatggagttgtgttaaccaagaaaatttccagttacccccatccaagactgatgtaaccaattgatgtagcttcttttataaatatttaggaacttgcttttaaggttttttttttttttgatgatgggttgcttttaagtaattttacatcctctaattatttttttcttaaatatgggattaaattgattgttacttgttgaagctaaaaaaggtttataatgttatggactaaattgatgttgtattgatttattggttcaactaaaataagaatataatggtaacacaataataatatcatttactcgtaaattattcttggtataatttttaaaatgattattataaaaatcaacaaaattattatatatgatgagttataattagatgaggtatatattttacaccgtgaatgtttccttattttcttaaaaataaaatgatggtaaaccttaaatcctatagtagcgctaaactaggttaagcttgcaactcttattcgctaacctggtgacaacagaactcttttgtttggacatttgcctagtaaagattagaagaggtccacaatggatggaaaggtacagttatacttctatttcggtaacttttagaatatttggcaaaattctcactaaacttgtagaatactttattcgttaaatagtacagttatctttttttttcaatgcaaaataatttaattgtcgaacataactttcaagagataaatgatttctacttacacggggaggataattgaatgtgggatttttttttattttacttctttagttctttatgggaaagaacttttaattaattcagaattcgatcataatttcgttaaagatcaaatatcaaatgattcaatcttaattttaatacattaattatttattataacgtgatttgatctcatattttttctatggtcaataaaatattggctaaatgatacgtgtagtcttttatgttattgtttagatttaatttaattatttatcttttaaatttagtttcatttaatcattctgcccgtttaaaattaatgttgttaataattaacatatcga

SEQ ID NO: 12 - DNA, 6746 bp, Glycine soja
tgtgtaaccaaagtctgttagtttaaaaaaaaattaattgtacccaaaaaataatataagtataaattggtacccaaaaaataatataagtatatatacatatataggtcggtctatcaggcttataaggctttctaataagcctaagtctggtttatttaatttaataggcttttaaaaaagtttgaacctaacattttaattaaataagtcagcccaggtcagattttatgtaggccaagtcgtagacccctgtaggccggcttagcttattctcacccctaattaagacaagctgtaggtcggcccctgttcatgttgttcatcaattattgtttaaccgagttaaaggagatggtcaattttacagtagtaaatatgacaagaaatacacgaaatttgtgtttaagtattgaaggtcttccatttttttgaatccaccatcctaaaactaatagtgtctaattttcccgtaacaatttcttttgtataacattcaaataaatgtggttgacgatatatatttagcttgcaaatttcctaaaaattattgctccaaacttagtatgcacttaggattggattgtgtcattattgtacttggcaagggtacatgtgttaattatatgcacttaagcgtgtcattgtacttggctacttaaaattgttttttgttcctcggatgatgttgatgagttataaaaaagaaatatatgccttgaaggaaagaaagtttatttttacggtttatgttaaaatacaatactttcaactatgtagattgtggagcaaacaagctttcagtttctctatcgtgttccgatgacacgttaataatgaacttttcctataaacatcaattagaagatgttgattttgttggtcaattggtgttattgttgtgactactttatattttcgttagattccaagcattatttccgtctatagggtttgctcttaactgattgttttgagttattaattattcaagttgatttgattggaatagtaaatctggatctattatgtaatatattagggacgaaatcttgcacattgagaatgatgtagtctcctcaaaattttgataaccattgttttcgagtgatagatgttgtctatgtcaaggttgtcgatgaaattagcaatgatggacagactttgtcgaacaagttttgtaatgccatcacagaaaagcatctagaagtactgaaaaacattttttttaacatatacttttacaaaatcaagagaatgaataattaaatatgaaataagaattacaaaaattttatatttttttaataaatttcaaatcaataataagaaagaatgggataaaattaaagaatatgttggaagatgtgttattagtatttctgtgcataagaatgcttccaaaattaaactctttccccaaaattgtatgagtaaaatttcaaatgtctcgttttaaatataacataagctaaattaaacccagcttagtaaaaaaaatgcatttccgtaaatttcgatgtccaaacaattgtaatgacttaaactcagaacaaaacctatttttataccatgtaaattcttacacattataatcgtatgtttatccatccatgcaccccaacaaatgaagaaaaggaataaaaaaaaaggtgaatttgccaaaaacaaattaatgggatgctaacattaacataaggcggcatagggcatcatttaattgtgaataaaaaggcttggtcaacccaatttcttctcctagttactagttattaaccatgtcatttggcatatatataataatccatatccatataaaaatacatgtcccaaaaattgaattccactacaccccaccaaagtggtgaattaatggccaaacgccactctcgcgttgacacccgcgagtgccaggcttgacgactctacgtacaacctcatcaattcctctatatatgcacataactaactttctctgtttttgacaccctcatacaccccatcatcttagctaacacaacacagcatacacaacttctctctctctactactttctttttatgtcaccttcttgatgtctttaatttgttttcttttttagaaaaaaattaatcctttttaaggctttaatttcctaagcatgcaatattattgtttttagtcaccttcaagttgaggaacatatacacgtttgtgaccacaccaaattccacttctttctaagtgtgtgtacatagattttgttttatatatatttgaggttcacccttcattgcctccttaattctgtgcaaaagaggattcatcaccaccatgttgcaggatgttgtccacccctcaacaccggctgagcaactccccattgtaatcacctcaagcttgttacatttcatgtctcgatcatgtatatttagtgttaatgcatgtattggaactaagctatatatgtatgtgtgtatgtatctttgtgtcatgcaatatttatgaccacagctaaatggtttccttctgggttttgtgatcgtgtgtaacttgacaagtttgtttggatcaagggcttgtttttttctttcaaaataatgatttcacactccaagagtgtgtaaaccttgggagggaagagaaagacaggaaagagaccccagaaaagaacaaaagaagtctaaaattgatgtacggattaatagcgtgtttgaaaacccattgagaagtaagaaaatctcatctgagatatgaaagttatgactattaacttcctaataaactcaagaaatcacatctgagatgtgaaaccaaacatgctcttacgaattgtagaaattttgatggtttttctctggggtactctttcttatattttgaggccaattattactaatttctctaggaattattcgatcaattgctccattcctgcattttaattctttccatgcatgaatcacgaatatggttttgttaatgtttgttggcatgttaattaatttcatcctaattagttctctgagaaacctgaacaatatttcccttatttggttatattcctggagctaagtggaagctgctagtgctattagccatcttagaaaacccacaagccatgctcactatttgtgggacggaacgttttctttcctctatttatatggttggagagcataacgtaattttattgaccgaacaataggaaggaatcaaagactccgtgaccgtgctgtcaactcttttgatatttattatcatccaatgggccctaccaccaaaatgtttaatgtcattttccaaggggcaaagttgtaaaaatgatgtaacatttccgtgatagtaatttagacatagctttcggttcttgtagaatatatatatctctttgggggcttgttgtgtaagctttgctaatttttgatattctttattcagttagatagggagaagaggtcttagtgacaaacaattcatatggatagcatatatagcattatggggttctcttcaagtggctttcaattcccatttaagttcaaattttccacttaaaatgaacaaaaataacatagcatgaggttccttgtttgtttttgcatacttgtatttcatactaacattggctaccttggctcctacctgtcacatgaaggcatagatgcacatcactttcaccaataaataacccaacaatgcaaaccctttaggaaaatttctcttcagtctattaccaaatatggacaaaactgactagactatgcaaccaaccacagtcatatgtagaattcttcgttggggcctttgcacatcgatttatattgttttcctcttgataaagaaggaagggggtgttgtggcagatactattatatcatttggcatgtctttcacttttgaatgcccactacatctacaagccaaagcttcaattgattagagaatattagcttttgaatttcttttctgttctaagtgatcagatcagactcatgtctatcaacaaaaagaggaattggattcagatttcaaacccatcataatgaagaaaaaaataaaaaaaataaactatagagaatcacgtttcctagattgtttataaagaagaggctttgttaagactattctcaggctgtgttttagtagtttatggtaaacatgttgttcacaatgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggtatgtgcaatttcgcaagccaattagagtttaatagacattcattgtctggtataaaagtttttacattatcaatcaatcagataccattgttgatataaattttaaaataattgttataataattaataatttaatgtacttgataatttgtgatttgataataatataaaaaaaatttacactgcattattatttatttttcctgtcgactgtcaataaactaaatgaaaattttcagttccaatgttcaatgtgttcagaataaggaaaaagaagtttaataatgctgcaaaggttactataccttgcagcagtgaagtttttattttaaaatagaagaggctttatcagaggtggacttttgggggaaagctcagggtccacaaatctctaaactataaactcataggtgccccatgaccatcaaatagtaggtagcacaagatatgagtccatttataaagtcacatgcattaaaaaatactataaatttggcctagcaagaaggaagaaccactttcatccaaaagaaaaatagaaaaaaggataataaactgtagcatcattagatagaaagacccacttcaagggtggcagtgttatatctctttctacagtctataaagttaatgtgcagtttttattgaataagtaagaaattgatctttaattataatttctctctcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaaggtactacatctgaacaccaacattaacaaacaaatttcaaatcttatactgttttacatgatttccaatctactgcatcaaccaagccttatgcatattttcaaaattcaactaatgatgcaattttttttatataaaaaaaatgcagtatgcttgccgcaaaactttggcggatagccggccccgggttagaggaaggtttgcaaagaatgatgactttggagagagccataaacaaggaagtagcaatcatgaagatgatgatgaagaggtaagattcccttaatcggatactgttgttcaacttgccttagtctaaaaattaaaatacaaaaaaattcccgatcacttttaccttttcaattatttgatggcataattccttgatgttatattccttccattttttgtacttgcagataattgtgaaagaagatgatgatatggttgattcctcagatatctttgcacatatcagcggagtgaactctttcaaatgcaactattccatccagtccttgatttgaattaaattattagtttgactagtgaaagcttatttatataattagcttctgtagattaattttggtaggacacttttcccatcccggttctctaaaatccgggtttagtggtttgagtaaactgaataaatggggtcaaaataaatataccaataagttaagtgagttagaaacgtacagaaattggaaactgtatacatttttgcagatatatattatctttttcattaagttgtaccagaacatggagttgtgttaaccaagaaaatttccagttacccccatccaagactgatgtaaccaattgatgtagcttcttttataaatatttaggaacttgcttttaaggttt

SEQ ID NO: 13 - DNA, 20 bp, Artificial Sequence
  Synthetic
gtgccgcaaaattagagaga

SEQ ID NO: 14 - DNA, 20 bp, Artificial Sequence
  Synthetic
gtatgcttgccgcaaaactt

SEQ ID NO: 15 - DNA, 20 bp, Artificial Sequence
  Synthetic
ggcacctgtggctgagctga

SEQ ID NO: 16 - DNA, 20 bp, Artificial Sequence
  Synthetic
gagtgtcaaagaggatggac

SEQ ID NO: 17 - DNA, 20 bp, Artificial Sequence
  Synthetic
gggtattgtatggaccagca

SEQ ID NO: 18 - DNA, 20 bp, Artificial Sequence
  Synthetic
ggcagtttgggataacccga

SEQ ID NO: 19 - DNA, 20 bp, Artificial Sequence
  Synthetic
gatgtcatgagaactacgca

SEQ ID NO: 20 - DNA, 20 bp, Artificial Sequence
  Synthetic
gtggatccagttcacttact

SEQ ID NO: 21 - DNA, 20 bp, Artificial Sequence
  Synthetic
ggcataagggccaccggtga

SEQ ID NO: 22 - DNA, 20 bp, Artificial Sequence
  Synthetic
gacgcacaataacctgaccc

SEQ ID NO: 23 - DNA, 32 bp, Artificial Sequence
  Synthetic
acgtaagcgcttacgcacgtaagcgcttacgc

SEQ ID NO: 24 - DNA, 1251 bp, Artificial Sequence
  Synthetic
atgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaagtatgcttgccgcaaaactttggcggatagccggccccgggttagaggaaggtttgcaaagaatgatgactttggagagagccataaacaaggaagtagcaatcatgaagatgatgatgaagagataattgtgaaagaagatgatgatatggttgattcctcagatatctttgcacatatcagcggagtgaactctttcaaatgcaactattccatccagtccttgatttga

SEQ ID NO: 25 - protein, 416 aa, Artificial Sequence
  Synthetic
MLICHHSLQDEISGPISARIFELCDPDFFPHTLQNSEVTSSSNCCHEEKSSYATTISPPLDVVDNNKFNINSNSSNIVTTTSSSTTTTSTTTNNNNNATNGNNLSIFFDTQDEIDNDISASIDFSSSPSFVVPPLLPISTQQDQFDFPSAQPQVQLSTAAGSILTGLSHYPTDPVIAPLIGAPLPSVFDDDCISSIPSYVPLNPSSPSCSYLSPGIGVYMPPPGSLNTALSADSSGLFGGNILLGSELQAHELDYQGENGGMYCTDSIQRVFNSPDLQALGNESQKLVAGAGSSATLAPEISHLEDSTLKVGKLSVEQRKEKIHRYMKKRNERNFSKKIKYACRKTLADSRPRVRGRFAKNDDFGESHKQGSSNHEDDDEEIIVKEDDDMVDSSDIFAHISGVNSFKCNYSIQSLI